(19) United States
(12) Patent Application Publication
(10) Pub. No.: US 2012/0183493 A1
(43) Pub. Date: Jul. 19, 2012

(54) NOVEL POLYMERS

(75) Inventor: Keith McCormack, Greater London (GB)

(73) Assignee: PharmaPatents Global Ltd., Cheshire (GB)

(21) Appl. No.: 13/319,453

(22) PCT Filed: May 11, 2010
(86) PCT No.: PCT/GB2010/050769

§ 371 Date: Mar. 20, 2012

(30) Foreign Application Priority Data
May 11, 2009 (GB) 0908053.2

(51) Int. Cl. A61K 31/787; A61P 17/02; A61P 43/00; C08G 69/08

(52) U.S. Cl. 424/78.36; 528/321

(57) ABSTRACT

The present invention relates to polymers comprising monomer units derived from captopril, and their use in the treatment or prevention of collagen deposition, fibrosis, scars, burns, or unwanted tissue formation. The present invention further relates to processes for the preparation of said polymers and to pharmaceutical compositions and medical products comprising the polymers. Also encompassed by the present invention are polymers comprising monomer units derived from other ACE inhibitors such as zofenopril, alacepril, rentiapril or pivalopril, or from vasopeptidase inhibitors such as fasidotrilat, omapatrilat or ilepatril.

### FIELD OF THE INVENTION

The present invention relates to polymers comprising monomer units derived from captopril, and their use in the treatment or prevention of collagen deposition, fibrosis, scars, burns, or unwanted tissue formation. The present invention further relates to processes for the preparation of said polymers and to pharmaceutical compositions and medical products comprising the polymers. Also encompassed by the present invention are polymers comprising monomer units derived from other ACE inhibitors such as zofenopril, alacepril, rentiapril or pivalopril, or from vasopeptidase inhibitors such as fasidotrilat, omapatrilat or ilepatril.

### BACKGROUND OF THE INVENTION

Captopril is a highly-specific competitive inhibitor of the angiotensin-I converting enzyme (ACE). It has the chemical structure:

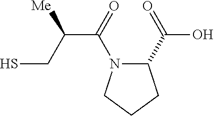

It is widely available and was the first ACE inhibitor to be developed. It is licensed for the treatment and management of hypertension, heart failure, myocardial infarction and type I diabetic nephropathy.

The present invention however relates to the use of ACE inhibitors for the treatment and prevention of collagen deposition, fibrosis, scars, burns and/or unwanted tissue formation.

Fibrosis can be defined as the overgrowth, hardening and/or scarring of various tissues and is attributed to excess deposition of extracellular matrix components including collagen.

Fibrosis is the end result of chronic inflammatory reactions for example induced by a variety of stimuli that include persistent infections, autoimmune reactions, allergic responses, chemical insults and radiation. Although current therapies for fibrotic diseases such as idiopathic pulmonary fibrosis, liver cirrhosis, systemic sclerosis, progressive kidney disease and cardiovascular fibrosis typically target the inflammatory response, there is accumulating evidence that the mechanisms driving fibrogenesis are distinct from those regulating inflammation.

Essentially a scar is what happens when injured tissue does not regenerate. Scarring occurs after trauma, injury or surgery to any tissue or organ in the body. As such, scars are a consequence of a repair mechanism that replaces the missing normal tissue with an extracellular matrix consisting predominantly of fibronectin and collagen types I and II. Accordingly, a scar replaces tissue regeneration and may be additionally considered to represent failed tissue regeneration.

In man and animals, scarring can cause major medical problems. For example in the eye, scarring can result in hazy vision or blindness; in the peripheral and central nervous system scarring is associated with a failure of neuronal reconnections with a resultant impairment in restoration of neuronal function; in the gastrointestinal tract strictures and adhesions caused by scarring can give rise to serious or life-threatening conditions; in the reproductive organs scarring can result in infertility; and in ligaments and tendons scarring can impede mechanical function and restrict the range of movement of the affected limb.

Not surprisingly, the skin represents the most frequently injured tissue, and dermal scarring after mechanical injury, trauma (from a variety of causes) and notably surgery invariably results in some degree of adverse medical outcome that includes impairment or loss of function, restriction of movement (associated with contractures over joints), restriction of growth and importantly poor aesthetics and psychological effects (especially following burns). Consequently on the skin, scars can have a dramatic effect upon patients, irrespective of whether or not the scars are hidden by clothing.

In addition to the effects of scars following injury, trauma and surgery to all tissues, scars impact upon function and quality of life in many acute and chronic fibrotic disorders that include myocardial infarct, drug-induced gingival overgrowth/hyperplasia, glomerulonephritis and pulmonary fibrosis for example, which share many of the cellular and molecular mechanisms common to scarring.

Teleologically, a scar can be seen as a predictable and robust response to injury and trauma that increases the probability of survival. Arguably, a diminished scarring response combined with a diminished regenerative capacity (as an alternative to scarring) would not represent a favourable trait, and such animals would not survive. Nature can be seen as less concerned about the consequences of scarring, such as disfigurement, that ordinarily do not pose an immediate threat to survival and continuity of the species.

Consistent with this teleological argument, it is not unreasonable to assume that scarring is an inevitable consequence of injury and by inference represents an evolutionarily optimized endpoint. However, this prevalent view is intellectually at odds with the results of experimental manipulations and observations. Apart from the numerous disadvantages associated with scar tissue, a scar is generally weaker than normal tissue, a fact that probably reflects the loss of alignment of extracellular matrix in a scar, by comparison with normal tissue (Lancet, 1992, vol. 339, pp. 213-214; J Cell Sci, 1994, vol. 107, pp. 1137-1157; J Cell Sci, 1995, vol. 108, pp. 985-1002; J Orthop Res, 1995, vol. 13(2), pp. 157-65; Clin Orthop Relat Res, 1997, vol. 337, pp. 272-80; Front Biosci, 2003, vol. 8, pp. s1240-8; Am J Sports Med, 2008, vol. 36(7), pp. 1290-7; J Refract Surg, 2005, vol. 21(5), pp. 433-45; J Sci Med Sport, 1999, vol. 2(3), pp. 190-210; J Orthop Res, 2009, vol. 27(3), pp. 400-7; Can J Surg, 1998, vol. 41(6), pp. 425-9).

Although there has been considerable attention upon preventing, improving and reversing scarring, most therapies have effects that are unpredictable and largely ineffective.

The effects of the ACE, renin-angiotensin system and the angiotensin receptors on the cardiovascular system, via their effects on smooth muscle tone for example, are well documented. However it has also been demonstrated that these enzymes and receptors have an additional role in mediating fibroblast proliferation. Consequently the application of ACE inhibitors to reduce collagen deposition, fibrosis and/or scar formation has become an area of increasing interest. The beneficial effects of ACE inhibitors in modelling/remodelling fibrous tissue formation in various injured circulatory organs, notably the heart, have been known for some time. Only more recently however has the much wider use of ACE inhibitors in inhibiting collagen and fibrous tissue formation in other organs, including the skin, begun to be contemplated.

Angiotensin II has been shown to be a key mediator in the development of fibroblast proliferation, fibrosis and scar formation. For instance, Ohuchi et al. (Comp Biochem Physiol C Toxicol Pharmacol, 2002, vol. 132(4), pp. 451-60; Comp Biochem Physiol C Toxicol Pharmacol, 2004, vol. 137(3), pp. 281-9) demonstrated that the anticonvulsant, phenyloin, and the antihypertensive calcium channel blocker, nifedipine, induced proliferation of guinea pig gingival fibroblast cells. Immunohistochemical experiments showed that the induced proliferation of gingival fibroblasts was associated with an increase in the immunostaining intensities of immunoreactive angiotensin (AT) II. The antihypertensive drug, captopril, an angiotensin converting enzyme (ACE) inhibitor (that blocks the formation of AT II), reduced these enhanced immunostaining intensities to control levels and inhibited the development of proliferation. These investigators concluded that in some part, phenyloin- and nifedipine-induced gingival fibroblast proliferation is mediated through the induction of AT II, via effects at the AT II type 1 (AT 1) receptor subtype.

These findings are consistent with the later findings of Ohuchi et al. (Comp Biochem Physiol C Toxicol Pharmacol, 2004, vol. 137(3), pp. 281-9) which demonstrate that gingival fibroblasts contain both AT 1 and 2 receptor subtypes for AT II. The authors added that this data supports the view that stimulation of AT 1 receptors by AT II results in the proliferation of fibroblasts. More recently, Santos et al. (J Periodontol, 2009, vol. 80(1), pp. 130-9) confirmed the existence of a renin-angiotensin system in gingival tissue that is capable of the local production of AT II via ACE.

In relation to fibrosis, a number of studies have been performed. For instance, Song et al. (Gastroenterol Hepatol, 2006, vol. 21(8), pp. 1250-6) showed that perindopril (an ACE inhibitor) and valsartan (a specific AT II receptor antagonist acting at the AT 1 receptor) can ameliorate progression of experimental hepatic fibrosis in rats. These results indicate an important role for AT II in the pathogenesis of hepatic fibrosis.

Similarly, AT II has been demonstrated to favour the in vitro and in vivo development of cardiac fibrosis (Cardiovasc Res, 1995, vol. 30(4), pp. 537-543; Circ Res, 1999, vol. 85(3), pp. 272-279; Circ Res, 2002, vol. 91(12), pp. 1119-1126; Circulation, 1993, vol. 88(6), pp. 2849-2861) through effects mediated via the activation of the AT 1 receptor (J Hypertens Suppl, 1997, vol. 15(6), pp. S13-19; Cardiovasc Res, 1994, vol. 28(11), pp. 1623-1628; J Renin Angiotensin Aldosterone Syst, 2001, vol. 2 (2), pp. 117-122).

Sun et al. (Cardiovasc Res, 2000, vol. 46(2), pp. 250-256) reported that pharmacologic intervention with ACE inhibitors is effective in attenuating scar tissue metabolic activity and minimizing adverse accumulation of fibrous tissue in non-infarcted myocardium. Some researchers have demonstrated that cultured myofibroblasts (obtained from four-week-old scar tissue of the left ventricle of adult rats with transmural myocardial infarction) are able to generate de novo AT I and AT II (J Mol Cell Cardiol, 1997, vol. 29(5), pp. 1375-1386). It was concluded that AT II may regulate myofibroblast collagen turnover and fibroblast tissue contraction in an autocrine and/or paracrine manner (J Mol Cell Cardiol, 1997, vol. 29(5), pp. 1375-1386; J Mol Cell Cardiol, 1997, vol. 29(8), pp. 2001-2012) and that myofibroblasts are the cells responsible for fibrous tissue formation in various injured organs, including the heart.

Although the role of AT II and the AT 1 receptor in the aetiogenesis of cardiac fibrosis is well documented, surprisingly it is only in recent times that the relationship between these mediators and cutaneous fibrous tissue remodelling has been explored.

Morihara et al. (J Am Acad Dermatol, 2006, vol. 54(2), pp. 251-257) studied ACE activity in normal skin, normally-healing wounded skin and pathologic scars. They observed that ACE activity in pathologic scars was significantly higher than in normal and wounded skin. Liu et al. (Zhonghua Zheng Xing Wai Ke Za Zhi, 2007, vol. 23(1), pp. 36-39) studied the expression of AT 1 and AT 2 receptors in human hypertrophic scars. They reported that positive staining signals of AT 1 and AT 2 receptors were found in fibroblasts from human hypertrophic scars. In cultured fibroblasts, stimulation with AT II resulted in an increase in DNA synthesis, which was inhibited by valsartan, an AT 1 receptor antagonist, but augmented by PD123319, an AT 2 receptor antagonist. Valsartan or PD123319 alone did not influence the proliferation of fibroblasts derived from hypertrophic scars. They concluded that both AT 1 and AT 2 receptors are expressed in the fibroblasts of hypertrophic scars, and that AT II regulates DNA synthesis in hypertrophic scar fibroblasts through a negative crosstalk between AT 1 and AT 2 receptors.

Acknowledging the role of AT II in mediating fibroblast proliferation and scar formation within cardiac tissue, Ardekani et al. (Wounds, 2008, vol. 20(4), pp. 101-106) report that topical application of captopril prevented the formation of hypertrophied scars in an animal model of pathologic scarring. They concluded that their study was the first animal study to report the effect of topical captopril upon scar formation, and that studies in humans were needed.

Iannelo et al. (Medscape General Medicine, 2006, vol. 8(4), p. 60) made a series of observations in two patients, including one of the co-authors. Firstly, the co-author developed an erythematous and painful postsurgical abdominal keloid scar after undergoing a left colectomy for colon adenocarcinoma. Four months later, after treatment with low dose enalapril (10 mg once daily) for mild arterial hypertension, her keloid scar rapidly improved and she eventually made a complete recovery. Secondly, a case is reported relating to a seventy year old female with diabetes who was affected by a postsurgical abdominal keloid scar of two years' duration. She was intentionally treated with the same low dose of enalapril, and, after six months of therapy, the authors reported that her scar showed marked improvement.

Steckelings et al. (Exp Dermatol, 2005, vol. 13(3), pp. 148-154; Br J Dermatol, 2005, vol. 153(5), pp. 887-93) studied the expression of angiotensin receptors in human skin following wounding. They studied punch biopsies from human skin ex vivo, and wound healing in sections of human cutaneous scars in vivo. They reported that enhanced expression of AT 1 and AT 2 was detectable as early as twenty-four hours after injury and lasted for up to three months. From these findings they concluded that angiotensin receptors AT 1 and AT 2 are up-regulated in human cutaneous wounds, giving further support to the concept that AT II plays a role even at an early stage during cutaneous wound healing.

Furthermore it is also speculated that additional beneficial effects on tissue regeneration and wound healing may be mediated via the angiotensin peptide Ang(1-7) [NorLeu(1-7)]. Studies have demonstrated that ACE inhibitors can increase the levels of Ang(1-7) by up to two orders of magnitude (see for instance Int J Biochem Cell Biol, 2003, vol. 35(6), pp. 792-801; and J Renin Angiotensin Aldosterone Syst, 2005, vol. 6(2), pp. 96-101). Meanwhile Rodgers and co-workers have shown that Ang(1-7) and the analogue NorLeu3-A(1-7) are able to accelerate dermal healing following wounding by reducing fibrosis and improving the collagen remodelling process (see J Pept Res, 2005, Suppl. 1, pp. 41-7; Wound Repair Regen, 2005, vol. 13(3), pp. 309-17; Plast Reconstr Surg, 2003, vol. 111(3), pp. 1195-206; and Exp Dermatol, 2003, vol. 12(6), pp. 784-90). Thus without wishing to be bound by theory it is believed that the beneficial effects of ACE inhibitors such as captopril may be observed both as a result of their ability to attenuate the effects of AT II and as a result of their ability to increase levels of Ang(1-7).

ACE inhibitors are conventionally delivered orally in tablet form. For cardiovascular applications, such as the treatment of hypertension or the prevention of restenosis, the use of a biodegradable polymer in which the ACE inhibitor is admixed to give injectable microparticles (see for example Mandal et al., Drug Development and Industrial Pharmacy, 1998, vol. 24(7), pp. 623-629) or in which the ACE inhibitor is encapsulated on the surface of a stent (see for example EP 1 319 416) has also been suggested. To date, however, delivery methods and formulations that are optimised for the treatment or prevention of collagen deposition, fibrosis, scars, burns and/or unwanted tissue formation have not even been considered.

### SUMMARY OF THE INVENTION

The present invention is based on the realisation that polymers that comprise ACE inhibitors such as captopril as part of their chemical structure are particularly useful for the treatment or prevention of collagen deposition, fibrosis, scars, burns and/or unwanted tissue formation. Such polymers offer the advantages that they allow for the delivery of the ACE inhibitor directly to the point of need, that they allow for the sustained release of the ACE inhibitor in a more controlled fashion than by mere admixture or encapsulation, and that they allow for a much higher loading of the ACE inhibitor within the polymer.

Furthermore, by incorporation of the ACE inhibitor into a polymer in the manner outlined below, and subsequent use of that polymer in for example the manufacture of a suture or surgical mesh, reliable scar-free or essentially scar-free tissue regeneration can be achieved without the need to subsequently remove the suture or mesh due to its inherent biodegradability.

The polymers incorporating —C(O)S— linkages are also believed to biodegrade at modified rates compared to those with —C(O)O— linkages, whilst still degrading sufficiently slowly to allow for the use of the polymers as sustained release agents in vivo. Without wishing to be bound by theory, it is also believed that the nature of the rate modification may vary depending on the environment in which the polymers are placed. Thus the polymers of the invention may find particular application in environments which are less suited to other types of polymer.

A further advantage of the polymers of any of the following aspects of the present invention is that they obviate the need for hydroxyalkyl acids such as glycolic acid to be used as monomers. Without wishing to be bound by theory, it is believed that both glycolic acid and polyglycolic acid may invoke an inflammatory response stimulating fibrosis (see for instance Stroke, 2003, vol. 34(8), pp. 2031-7; Br J Urol, 1997, vol. 80(6), pp. 903-7; Fertil Steril, 1983, vol. 40(6), pp. 815-7; Tissue Eng, 2006, vol. 12(2), pp. 301-8; Mol Cells, 2008, vol. 26(6), pp. 625-30; Dermatol Surg, 1996, vol. 22(9), pp. 781-6; and Skin Therapy Lett, 2004, vol. 9(2), pp. 6-11). Thus, the development of polymers which do not incorporate a large amount of such acids is desirable.

Accordingly, a first aspect of the present invention relates to a polymer comprising a unit

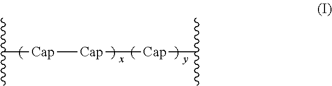

wherein x is an integer ≧1, y is 0 or 1, and -Cap- is a unit of formula II:

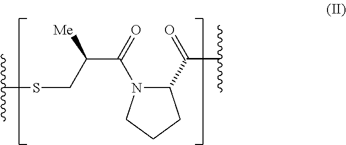

Thus it can be seen that where -Cap- is a unit of formula II, H-Cap-OH represents cap topril.

Preferably x is an integer from 1 to 100,000. More preferably x is an integer from 1 to 10,000. Most preferably x is an integer from 1 to 1,000.

In a first embodiment of the first aspect of the present invention, the polymer is a homo-polymer. Preferably in such an embodiment, the polymer has the formula Ia:

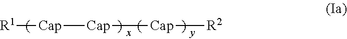

wherein R¹ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and

R² is —R³, —OR³, —SR³ or —N(R³)₂, wherein each R³ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R³ groups together with the atom or atoms to which they are attached may form a cyclic hydrocarbyl group which may optionally be substituted and which may optionally include one or more heteroatoms N, O or S in its carbon skeleton.

Preferably R¹ is hydrogen or contains from 1 to 12 carbon atoms. More preferably R¹ is hydrogen or contains from 1 to 6 carbon atoms. Even more preferably R¹ is hydrogen or an acyl group. Most preferably R¹ is hydrogen.

Preferably R² is —OR³ or —SR³. More preferably R² is —OR³. Preferably each R³ is independently hydrogen or contains from 1 to 12 carbon atoms. More preferably each R³ is independently hydrogen or contains from 1 to 6 carbon atoms. Even more preferably each R³ is independently hydrogen or an alkyl group. Most preferably each R³ is hydrogen.

In a particularly preferred embodiment, R¹ is hydrogen and R² is —OH.

Preferably in the first embodiment of the first aspect of the present invention, x is an integer from 1 to 100,000. More preferably x is an integer from 10 to 10,000. Most preferably x is an integer from 100 to 1,000.

Alternately, x may be an integer from 1 to 1,000. Preferably x is an integer from 2 to 100, or from 2 to 20. Optionally x is an integer from 3 to 10. Optionally x is an integer from 3 to 5.

In a second embodiment of the first aspect of the present invention, the polymer is a copolymer, such as a periodic copolymer, a random copolymer or a block copolymer, formed with one or more additional polymeric substances. Where the copolymer is a block copolymer, it may optionally comprise as a subunit a homo-polymer according to the first embodiment of the first aspect of the present invention.

Preferably in the second embodiment of the first aspect of the present invention, one or more of the additional polymeric substances is a biodegradable polymer, such as one selected from the group consisting of poly-lactic acids, poly-lactides, polylactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof.

Alternatively or in addition, one or more of the additional polymeric substances may be a non-biodegradable polymer, such as one selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

Preferably in the second embodiment of the first aspect of the present invention, especially where the copolymer is a periodic copolymer, x is an integer from 1 to 1,000. Preferably x is an integer from 2 to 100, or from 2 to 20. Optionally x is an integer from 3 to 10. Optionally x is an integer from 3 to 5.

In a third embodiment of the first aspect of the present invention, any unit of formula (I) does not comprise Au(I). Preferably any unit of formula (I) does not comprise any Au atoms. More preferably any unit of formula (I) does not comprise any Cu, Ag or Au atoms. More preferably still any unit of formula (I) does not comprise any metal atoms.

A second aspect of the present invention relates to a polymer comprising a unit of formula III within the polymer backbone:

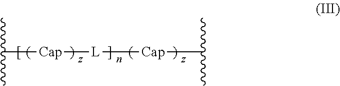

wherein n is an integer ≧1, each z is independently an integer ≧1, each L is independently any linking atom or group, and -Cap- is a unit of formula II:

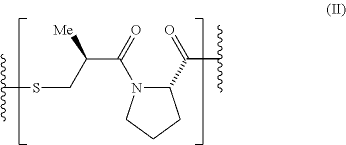

with the proviso that the unit of formula III does not comprise a -Cap-O— unit.

In one embodiment of the second aspect of the present invention, the polymer backbone does not comprise a -Cap-O— unit other than at a terminal position.

In another embodiment of the second aspect of the present invention, the polymer backbone and/or the unit of formula III does not comprise a —COO— group within 2 bonds of a -Cap- unit other than at a terminal position. Preferably the polymer backbone and/or the unit of formula III does not comprise a —COO— group within 3 bonds, 5 bonds or 10 bonds of a -Cap- unit other than at a terminal position. Most preferably the polymer backbone and/or the unit of formula III does not comprise a —COO— group other than at a terminal position.

In yet another embodiment of the second aspect of the present invention, each bond within the polymer backbone and/or the unit of formula III is more resistant to hydrolysis than the ester bond in H-Cap-OEt.

Preferably each -Cap- unit within the polymer backbone and/or the unit of formula III, other than a terminal -Cap- unit, is part of a -Cap-S— unit.

In one embodiment of the second aspect of the present invention, at least one -Cap- unit is not at a terminal position. Preferably, at least two, three, five, ten or twenty -Cap- units are not at terminal positions. Optionally no -Cap- units are at terminal positions.

In another embodiment of the second aspect of the present invention, any unit of formula (III) does not comprise Au(I). Preferably any unit of formula (III) does not comprise any Au atoms. More preferably any unit of formula (III) does not comprise any Cu, Ag or Au atoms. More preferably still any unit of formula (III) does not comprise any metal atoms.

In yet another embodiment of the second aspect of the present invention, the polymer backbone does not comprise Au(I). Preferably the polymer backbone does not comprise any Au atoms. More preferably the polymer backbone does not comprise any Cu, Ag or Au atoms. More preferably still the polymer backbone does not comprise any metal atoms.

In another embodiment of the second aspect of the present invention, the polymer has the formula IIIa:

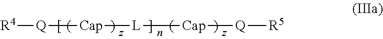

wherein each Q is independently L or a chemical bond;

R⁴ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and

R⁵ is —R⁶, —OR⁶, —SR⁶ or —N(R⁶)₂, wherein each R⁶ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R⁶ groups together with the atom or atoms to which they are attached may form a cyclic hydrocarbyl group which may optionally be substituted and which may optionally include one or more heteroatoms N, O or S in its carbon skeleton.

Preferably R⁴ is hydrogen or contains from 1 to 12 carbon atoms. More preferably R⁴ is hydrogen or contains from 1 to 6 carbon atoms. Even more preferably R⁴ is hydrogen or an acyl group. Most preferably R⁴ is hydrogen.

Preferably R⁵ is —OR⁶ or —SR⁶. More preferably R⁵ is —OR⁶. Preferably each R⁶ is independently hydrogen or contains from 1 to 12 carbon atoms. More preferably each R⁶ is independently hydrogen or contains from 1 to 6 carbon atoms. Even more preferably each R⁶ is independently hydrogen or an alkyl group. Most preferably each R⁶ is hydrogen.

In a particularly preferred embodiment, R⁴ is hydrogen and R⁵ is —OH.

In one embodiment each Q is a chemical bond. In an alternate embodiment each Q is L. In another embodiment the Q connected to R⁴ is a chemical bond and the Q connected to R⁵ is L. In yet another embodiment the Q connected to R⁵ is a chemical bond and the Q connected to R⁴ is L.

In one embodiment of the second aspect of the present invention, n is an integer from 1 to 100,000. Preferably n is an integer from 10 to 10,000. Most preferably n is an integer from 100 to 1,000.

In another embodiment of the second aspect of the present invention, each z is independently an integer from 1 to 100, or from 1 to 50, or from 1 to 25. Preferably each z is independently an integer from 1 to 10. In one embodiment each z is 1.

Preferably, each L has a chain length of from 1 to 100 chemical bonds. More preferably, each L has a chain length of from 2 to 20 chemical bonds. Most preferably each L has a chain length of from 3 to 6 chemical bonds.

In one embodiment of the second aspect of the present invention, no L comprises Au(I). Preferably no L comprises any Au atoms. More preferably no L comprises any Cu, Ag or Au atoms. More preferably still no L comprises any metal atoms.

In another embodiment of the second aspect of the present invention, each L comprises one or more monomer units from a biodegradable polymer, such as one selected from the group consisting of poly-lactic acids, poly-lactides, polylactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof.

In an alternate or additional embodiment of the second aspect of the present invention, each L comprises one or more monomer units from a non-biodegradable polymer, such as one selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

In another embodiment of the second aspect of the present invention, each L has the

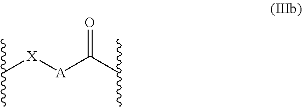

wherein X is —S— or —NR⁷—;

A is a chemical bond or an alkylene, alkenylene, alkynylene, arylene, arylalkylene, arylalkenylene, arylalkynylene, alkylarylene, alkenylarylene or alkynylarylene group, each of which may optionally be substituted, and each of which may optionally include one or more heteroatoms N, O or S in its carbon skeleton;

R⁷ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and

wherein R⁷ and any substituent of A together with the atoms to which they are attached may form a cyclic hydrocarbyl group which may optionally be substituted and which may optionally include one or more additional heteroatoms N, O or S in its carbon skeleton.

Preferably X is —S—.

Preferably A has from 1 to 20 carbon atoms. More preferably A has from 2 to 10 carbon atoms.

Optionally, A is —(CH₂)ₘ— wherein m is an integer from 1 to 50. Preferably m is an integer from 1 to 20. More preferably m is an integer from 2 to 10.

In yet another embodiment of the second aspect of the present invention, each L comprises one or more -Cap- units as defined in accordance with the fourteenth aspect of the present invention. Thus each L may comprise for instance a unit of formula IIa, IIb, IIc, IId, IIe, IIf, IIg, IIh, IIi, IIj, IIk, IIm or IIn. Preferably each L comprises a unit of formula IIe, IIi, IIm or IIn.

In one embodiment according to either the first or the second aspect of the present invention, the polymer comprises from 2 to 200,000-Cap- units within the polymer and/or polymer backbone. Preferably the polymer comprises from 5 to 10,000-Cap- units within the polymer and/or polymer backbone. More preferably the polymer comprises from 10 to 1,000-Cap- units within the polymer and/or polymer backbone. Optionally the polymer comprises from 20 to 100-Cap- units within the polymer and/or polymer backbone.

In another embodiment according to either the first or the second aspect of the present invention, the polymer is appended to, terminates or cross-links the backbone(s) of one or more base polymers.

A third aspect of the present invention relates to a polymer comprising one or more base polymers and one or more cross-linking groups wherein at least one cross-linking group comprises one or more -Cap- units of formula II:

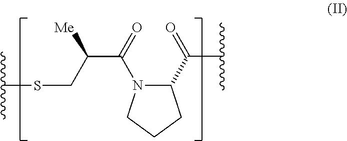

In one embodiment of the third aspect of the present invention, the cross-linking group comprises from 1 to 10-Cap- units. Preferably the cross-linking group comprises from 1 to 5-Cap- units. Most preferably the cross-linking group comprises 1, 2 or 3-Cap- units.

In another embodiment of the third aspect of the present invention, the cross-linking group contains only -Cap- units.

In another embodiment of the third aspect of the present invention, the base polymers and/or the cross-linking groups do not comprise Au(I). Preferably the base polymers and/or the cross-linking groups do not comprise any Au atoms. More preferably the base polymers and/or the cross-linking groups do not comprise any Cu, Ag or Au atoms. More preferably still the base polymers and/or the cross-linking groups do not comprise any metal atoms.

In an alternate embodiment of the third aspect of the present invention, the cross-linking group further comprises one or more linking groups L′, wherein each L′ is independently any atom or group. Preferably such a cross-linking group has the formula IV:

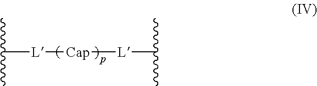

wherein p is an integer ≧1.

Preferably p is an integer from 1 to 10. More preferably p is an integer from 1 to 5. Most preferably p is 1, 2 or 3.

In one embodiment, each L′ has a chain length of 1 to 100 chemical bonds. Preferably each L′ has a chain length of 2 to 20 chemical bonds. More preferably each L′ has a chain length of 3 to 6 chemical bonds.

In another embodiment of the third aspect of the present invention, no L′ comprises Au(I). Preferably no L′ comprises any Au atoms. More preferably no L′ comprises any Cu, Ag or Au atoms. More preferably still no L′ comprises any metal atoms.

In another embodiment, each L′ comprises one or more monomer units from a biodegradable polymer, such as one selected from the group consisting of poly-lactic acids, poly-lactides, polylactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof.

In an additional or alternate embodiment, each L′ comprises one or more monomer units from a non-biodegradable polymer, such as one selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

In yet another embodiment, each L′ has the formula IVa:

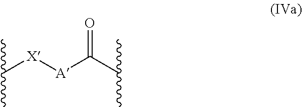

wherein X′ is —O—, —S— or —NR⁸—;

A′ is a chemical bond or an alkylene, alkenylene, alkynylene, arylene, arylalkylene, arylalkenylene, arylalkynylene, alkylarylene, alkenylarylene or alkynylarylene group, each of which may optionally be substituted, and each of which may optionally include one or more heteroatoms N, O or S in its carbon skeleton;

R⁸ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and

wherein R⁸ and any substituent of A′ together with the atoms to which they are attached may form a cyclic hydrocarbyl group which may optionally be substituted and which may optionally include one or more additional heteroatoms N, O or S in its carbon skeleton.

Preferably X′ is —S—.

Preferably A′ has from 1 to 20 carbon atoms. More preferably A′ has from 2 to 10 carbon atoms.

Optionally, A′ is —(CH₂)_{m′}—, wherein m′ is an integer from 1 to 50. Preferably m′ is an integer from 1 to 20. More preferably m′ is an integer from 2 to 10.

In yet another embodiment of the third aspect of the present invention, each L′ comprises one or more -Cap- units as defined in accordance with the fourteenth aspect of the present invention. Thus each L′ may comprise for instance a unit of formula IIa, IIb, IIc, IId, IIe, IIf, IIg, IIh, IIi, IIj, IIk, IIm or En. Preferably each L′ comprises a unit of formula IIe, IIi, IIm or IIn.

Preferably the cross-linking groups of the third aspect of the present invention are biodegradable. Such cross-linking groups offer the advantage that they can be tailored to degrade at a different rate to the base polymer, changing the physical properties of the polymer as a whole as the cross-linking groups biodegrade. For example, a rigid mesh made from such a polymer may become more flexible as the captopril in the cross-linking groups is released and the wound to which it is applied heals.

In any aspect of the present invention comprising a base polymer, in one embodiment one or more of the base polymers are biodegradable polymers, such as those selected from the group consisting of poly-lactic acids, poly-lactides, polylactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof.

Alternatively or in addition, one or more of the base polymers may be non-biodegradable polymers, such as those selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

In one embodiment of any of the first, second or third aspects of the present invention, the polymer is appended by or terminated with one or more groups selected from R⁹-Cap-, i.e. where -Cap- is a unit of formula II, R⁹-Cap- is

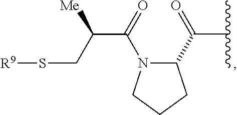

and -Cap-R¹⁰, i.e. where -Cap- is a unit of formula II, -Cap-R¹⁰ is

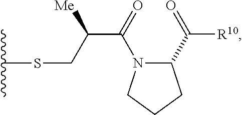

wherein R⁹ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
R¹⁰ is —R¹¹, —OR¹¹, —SR¹¹ or —N(R¹¹)₂ wherein each R¹¹ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R¹¹ groups together with the atom or atoms to which they are attached may form a cyclic hydrocarbyl group which may optionally be substituted and which may optionally include one or more heteroatoms N, O or S in its carbon skeleton.

Preferably R⁹ is hydrogen or contains from 1 to 12 carbon atoms. More preferably R⁹ is hydrogen or contains from 1 to 6 carbon atoms. Even more preferably R⁹ is hydrogen or an acyl group. Most preferably R⁹ is hydrogen.

Preferably R¹⁰ is —OR¹¹ or —SR¹¹. More preferably R¹⁰ is —OR¹¹. Preferably each R¹¹ is independently hydrogen or contains from 1 to 12 carbon atoms. More preferably each R¹¹ is independently hydrogen or contains from 1 to 6 carbon atoms. Even more preferably each R¹¹ is independently hydrogen or an alkyl group. Most preferably each R¹¹ is hydrogen.

In a particularly preferred embodiment, R⁹ is hydrogen and R¹⁹ is —OH.

In one embodiment of any of the first, second or third aspects of the present invention, the polymer further comprises one or more additional active pharmaceutical ingredients appended to, terminating or incorporated in the backbone of the polymer. Preferably one or more of the additional active pharmaceutical ingredients are ACE inhibitors, such as captopril, zofenopril, zofenoprilat, lisinopril, benazepril, benazeprilat, cilazapril, cilazaprilat, moexipril, moexiprilat, perindopril, perindoprilat, quinapril, quinaprilat, ramipril, ramiprilat, spirapril, spiraprilat, trandolapril, trandolaprilat, alacepril, desacetyl-alacepril, delapril, delaprilat, imidapril, imidaprilat, rentiapril, temocapril, temocaprilat, ceronapril, enalapril, enalaprilat, moveltipril, pivalopril, despivaloyl-pivalopril, fosinopril or fosinoprilat. More preferably one or more of the additional active pharmaceutical ingredients are captopril, alacepril, rentiapril, pivalopril, despivaloyl-pivalopril, zofenopril or zofenoprilat. More preferably still one or more of the additional active pharmaceutical ingredients are captopril, zofenopril or zofenoprilat.

One or more of the additional active pharmaceutical ingredients may also be selected from the group consisting of vasopeptidase inhibitors, i.e. compounds which inhibit both angiotensin converting enzyme and neutral endopeptidase. Exemplary vasopeptidase inhibitors suitable for use in the present invention include fasidotril, fasidotrilat, omapatrilat, sampatrilat, ilepatril (AVE 7688), des-acetyl ilepatril, CGS 35601, CGS 37808 and derivatives thereof. Preferably the vasopeptidase inhibitor is selected from fasidotrilat, omapatrilat, des-acetyl ilepatril, CGS 35601 and derivatives thereof.

One or more of the additional active pharmaceutical ingredients may also be selected from other inhibitors of the renin-angiotensin pathway including renin inhibitors such as aliskiren or remikiren; angiotensin II antagonists such as azilsartan, candesartan, eprosartan, irbesartan, losartan, olmesartan, tasosartan, telmisartan or valsartan; and/or an active peptide fragment of angiotensin I or II including Ang (1-7), i.e. NorLeu(1-7) or an analogue thereof such as NorLeu3-A(1-7).

One or more of the additional active pharmaceutical ingredients may also be selected from COX inhibitors including bendazac, etofenamate or fluproquazone; salicylates such as aspirin, aloxiprin, benorylate, diflunisal, ethenzamide, magnesium salicylate, methyl salicylate, salsalate, salicin, salicylamide or sodium salicylate; arylalkanoic acids such as diclofenac, aceclofenac, acemetacin, alclofenac, bromfenac, etodolac, indometacin, indometacin farnesil, nabumetone, oxametacin, proglumetacin, sulindac or tolmetin; 2-arylpropionic acids such as ibuprofen, alminoprofen, benoxaprofen, carprofen, dexibuprofen, dexketoprofen, fenbufen, felbinac, fenoprofen, flunoxaprofen, flurbiprofen, ibuproxam, indoprofen, ketoprofen, ketorolac, loxoprofen, miroprofen, naproxen, oxaprozin, pirprofen, suprofen, tarenflurbil or tiaprofenic acid; N-arylanthranilic acids such as mefenamic acid, flufenamic acid, meclofenamic acid or tolfenamic acid; pyrazolidine derivatives such as phenylbutazone, ampyrone, azapropazone, clofezone, kebuzone, metamizole, mofebutazone, oxyphenbutazone, phenazone or sulfinpyrazone; oxicams such as piroxicam, droxicam, lornoxicam, meloxicam, tenoxicam or ampiroxicam; selective COX-2 inhibitors such as celecoxib, deracoxib, etoricoxib, firocoxib, lumiracoxib, parecoxib, rofecoxib or valdecoxib; sulfonanilides such as nimesulide; and COX-inhibiting nitric oxide donators such as naproxcinod.

One or more of the additional active pharmaceutical ingredients may also be selected from glucocorticoids including non-halogenated glucocorticoids such as cordsone, hydrocortisone, budesonide, ciclesonide, cortivazol, deflazacort, hydrocortisone aceponate, hydrocortisone buteprate, hydrocortisone butyrate, meprednisone, methylprednisolone, methylprednisolone aceponate, prednicarbate, prednisolone, prednisone, prednylidene or rimexolone; halogenated glucocorticoids such as beclometasone, betamethasone, cloprednol, dexamethasone, fluticasone, flunisolide, mometasone furoate, paramethasone or triamcinolone; or selected from osteopontin inhibitors such as the antisense (AS) oligodeoxynucleotides described in J Exp Med, 2008, vol. 205(1), pp. 43-51.

In another embodiment of any of the first, second or third aspects of the present invention, the polymer is entirely biodegradable.

In another embodiment of any of the first, second or third aspects of the present invention, the decomposition products of the polymer are only active pharmaceutical ingredients and/or pharmaceutically acceptable decomposition products.

In yet another embodiment of any of the first, second or third aspects of the present invention, the polymer has an average molecular weight of from 400 to 5,000,000. Preferably the polymer has an average molecular weight of from 1,000 to 1,000,000. Most preferably the polymer has an average molecular weight of from 10,000 to 400,000.

In an alternate embodiment of any of the first, second or third aspects of the present invention, the polymer has an average molecular weight of from 350 to 10,000. Preferably the polymer has an average molecular weight of from 1,200 to 2,000. Most preferably the polymer has an average molecular weight of about 1,600.

Preferably, in any of the first, second or third aspects of the present invention, less than 50% of the monomers of the polymer are glycolic acid. More preferably, less than 40%, less than 20%, less than 10% or less than 5% of the monomers of the polymer are glycolic acid. Most preferably none of the monomers of the polymer are glycolic acid.

Preferably, in any of the first, second or third aspects of the present invention, less than 50% of the monomers of the polymer are hydroxyalkyl acids. More preferably, less than 40%, less than 20%, less than 10% or less than 5% of the monomers of the polymer are hydroxyalkyl acids. Most preferably none of the monomers of the polymer are hydroxyalkyl acids.

A fourth aspect of the present invention relates to a process for synthesising a polymer according to any of the first, second or third aspects of the present invention, said process comprising the use of H-Cap-OH and/or

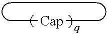

wherein q is an integer from 1 to 10, and/or protected derivatives thereof.

A fifth aspect of the present invention relates to a process for synthesising a polymer, said process comprising:
(a) the condensation of a —COOH group of either H-Cap-OH or a protected derivative thereof with a —SH group of another molecule; and/or
(b) the condensation of a —C(O)S— group of either

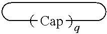

or a protected derivative thereof, wherein q is an integer from 1 to 10, with a —SH group of another molecule; and/or
(c) the condensation of a —SH group of either H-Cap-OH or a protected derivative thereof with a —COOH group of another molecule;
   wherein -Cap- is a unit of formula II:

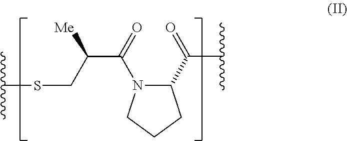

For the avoidance of doubt, the condensations specified above also include the condensations of salts of the groups, molecules or derivatives thereof. As used herein, it is also to be understood that the term “condensation” when applied to two or more groups means that a chemical bond is formed between at least one atom of each group such that the moieties to which the respective groups are attached become covalently bound to one another. Such a condensation need not be direct and may proceed for instance via thio-lactone ring opening to give an intermediate species, via chemical activation of the —COOH group and/or via the use of an enzyme.

In one embodiment of the fifth aspect of the present invention, any condensation between a —SH group of either H-Cap-OH or a protected derivative thereof and a —COOH group of another molecule is not a free-radical process. Preferably any condensation of the fifth aspect of the present invention is not a free-radical process.

In another embodiment of the fifth aspect of the present invention, any —COOH group involved in the condensation is not part of a poly-lactide-co-glycolide. Preferably any —COOH group involved in the condensation is not part of a polylactide, a polyglycolide or a poly-lactide-co-glycolide. More preferably any —COOH group involved in the condensation is not part of a lactide, a glycolide, a polylactide, a polyglycolide or a poly-lactide-co-glycolide.

In another embodiment of the fifth aspect of the present invention, any —COOH group involved in the condensation is not part of a peptide. Preferably any —COOH group involved in the condensation is not part of a peptide or an amino acid. More preferably any —COOH group involved in the condensation is not part of a protein, a peptide or an amino acid.

In yet another embodiment of the fifth aspect of the present invention, any —SH group involved in the condensation is not part of a peptide. Preferably any —SH group involved in the condensation is not part of a peptide or an amino acid. More preferably any —SH group involved in the condensation is not part of a protein, a peptide or an amino acid.

In one embodiment of the fifth aspect of the present invention, the condensation is between two molecules of H-Cap-OH, or protected and/or polymerised derivatives thereof.

In another embodiment of the fifth aspect of the present invention, the condensation is between one molecule of H-Cap-OH, or a protected and/or polymerised derivative thereof, and one molecule of

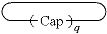

or a protected derivative thereof.

In yet another embodiment of the fifth aspect of the present invention, the condensation is between two molecules of

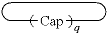

or protected derivatives thereof.

Preferably the process of the fifth aspect of the present invention is for synthesising a polymer according to any of the first, second or third aspects of the present invention.

In one embodiment of the fourth or fifth aspects of the present invention, the synthesis is achieved via chemical activation of a —COOH group.

In another embodiment of the fourth or fifth aspects of the present invention, the synthesis is achieved via enzymatic catalysis. Preferably a lipase enzyme is used such as a lipase from Candida antartica. Most preferably lipase B from Candida antartica is used such as that with CAS no. 9001-62-1 available as ‘Novozymes lipase B from Candida antartica’ from Codexis, Redwood City, Calif., USA.

Preferably when enzymatic catalysis is used, water is removed from the enzymes prior to use. Preferably the enzymes are stored over molecular sieves prior to use.

Preferably in any embodiment of the fourth or fifth aspects of the present invention q is 1 or 2. Most preferably q is 1, i.e. where -Cap- is a unit of formula II,

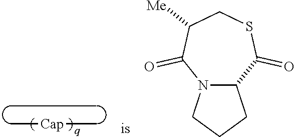

A sixth aspect of the present invention relates to a polymer produced by a process according to the fourth or fifth aspects of the present invention.

A seventh aspect of the present invention relates to a pharmaceutical composition comprising a polymer according to any of the first, second, third or sixth aspects of the present invention. Preferably the pharmaceutical composition further comprises one or more pharmaceutically acceptable excipients and/or one or more additional active pharmaceutical ingredients.

Preferably one or more of the additional active pharmaceutical ingredients are ACE inhibitors, such as captopril, zofenopril, zofenoprilat, lisinopril, benazepril, benazeprilat, cilazapril, cilazaprilat, moexipril, moexiprilat, perindopril, perindoprilat, quinapril, quinaprilat, ramipril, ramiprilat, spirapril, spiraprilat, trandolapril, trandolaprilat, alacepril, desacetyl-alacepril, delapril, delaprilat, imidapril, imidaprilat, rentiapril, temocapril, temocaprilat, ceronapril, enalapril, enalaprilat, moveltipril, pivalopril, despivaloyl-pivalopril, fosinopril or fosinoprilat. More preferably one or more of the additional active pharmaceutical ingredients are captopril, alacepril, rentiapril, pivalopril, despivaloyl-pivalopril, zofenopril or zofenoprilat. More preferably still one or more of the additional active pharmaceutical ingredients are captopril, zofenopril or zofenoprilat.

One or more of the additional active pharmaceutical ingredients may also be selected from the group consisting of vasopeptidase inhibitors, i.e. compounds which inhibit both angiotensin converting enzyme and neutral endopeptidase. Exemplary vasopeptidase inhibitors suitable for use in the present invention include fasidotril, fasidotrilat, omapatrilat, sampatrilat, ilepatril (AVE 7688), CGS 35601, CGS 37808 and derivatives thereof. Preferably the vasopeptidase inhibitor is selected from fasidotril, omapatrilat, sampatrilat, ilepatril (AVE 7688), CGS 35601 and derivatives thereof.

One or more of the additional active pharmaceutical ingredients may also be selected from other inhibitors of the renin-angiotensin pathway including renin inhibitors such as aliskiren or remikiren; angiotensin II antagonists such as azilsartan, candesartan, eprosartan, irbesartan, losartan, olmesartan, tasosartan, telmisartan or valsartan; and/or an active peptide fragment of angiotensin I or II including Ang (1-7), i.e. NorLeu(1-7) or an analogue thereof such as NorLeu3-A(1-7).

One or more of the additional active pharmaceutical ingredients may also be selected from COX inhibitors including bendazac, etofenamate or fluproquazone; salicylates such as aspirin, aloxiprin, benorylate, diflunisal, ethenzamide, magnesium salicylate, methyl salicylate, salsalate, salicin, salicylamide or sodium salicylate; arylalkanoic acids such as diclofenac, aceclofenac, acemetacin, alclofenac, bromfenac, etodolac, indometacin, indometacin farnesil, nabumetone, oxametacin, proglumetacin, sulindac or tolmetin; 2-arylpropionic acids such as ibuprofen, alminoprofen, benoxaprofen, carprofen, dexibuprofen, dexketoprofen, fenbufen, felbinac, fenoprofen, flunoxaprofen, flurbiprofen, ibuproxam, indoprofen, ketoprofen, ketorolac, loxoprofen, miroprofen, naproxen, oxaprozin, pirprofen, suprofen, tarenflurbil or tiaprofenic acid; N-arylanthranilic acids such as mefenamic acid, flufenamic acid, meclofenamic acid or tolfenamic acid; pyrazolidine derivatives such as phenylbutazone, ampyrone, azapropazone, clofezone, kebuzone, metamizole, mofebutazone, oxyphenbutazone, phenazone or sulfinpyrazone; oxicams such as piroxicam, droxicam, lornoxicam, meloxicam, tenoxicam or ampiroxicam; selective COX-2 inhibitors such as celecoxib, deracoxib, etoricoxib, firocoxib, lumiracoxib, parecoxib, rofecoxib or valdecoxib; sulfonanilides such as nimesulide; and COX-inhibiting nitric oxide donators such as naproxcinod.

One or more of the additional active pharmaceutical ingredients may also be selected from glucocorticoids including non-halogenated glucocorticoids such as cordsone, hydrocortisone, budesonide, ciclesonide, cortivazol, deflazacort, hydrocortisone aceponate, hydrocortisone buteprate, hydrocortisone butyrate, meprednisone, methylprednisolone, methylprednisolone aceponate, prednicarbate, prednisolone, prednisone, prednylidene or rimexolone; halogenated glucocorticoids such as beclometasone, betamethasone, cloprednol, dexamethasone, fluticasone, flunisolide, mometasone furoate, paramethasone or triamcinolone; or selected from osteopontin inhibitors such as the antisense (AS) oligodeoxynucleotides described in J Exp Med, 2008, vol. 205(1), pp. 43-51.

An eighth aspect of the present invention relates to a medical product comprising a polymer according to any of the first, second, third or sixth aspects of the present invention, or comprising a pharmaceutical composition according to the seventh aspect of the present invention.

Preferably the medical product is selected from the group consisting of grafts, stents, catheters, bone plates, dental implants, sutures, staples, surgical meshes, wound dressings, plasters, films such as cling-film, contact lenses, protective clothing, bone cements, implantable sensors, implantable drug delivery devices, cosmetic implants such as silicone implants, artificial joint or bone replacements and other implantable medical devices and prosthetics. Most preferably the medical product is selected from the group consisting of sutures, staples, surgical meshes, wound dressings, plasters and films such as cling-film.

A ninth aspect of the present invention relates to a polymer according to any of the first, second, third or sixth aspects of the present invention, or a pharmaceutical composition according to the seventh aspect of the present invention, or a medical product according to the eighth aspect of the present invention, for use as a medicament.

The medicament employed in the present invention can be administered by oral, parenteral (including intravenous, subcutaneous, intramuscular, intradermal, intratracheal, intraperitoneal, intraarticular, intracranial and epidural), transdermal, airway (aerosol), rectal, vaginal or topical (including buccal, mucosal and sublingual) administration. Topical and/or parenteral administration is however preferred.

For oral administration, the polymers of the invention will generally be provided in the form of tablets, capsules, hard or soft gelatin capsules, caplets, troches or lozenges, as a powder or granules, or as an aqueous solution, suspension or dispersion.

Tablets for oral use may include the polymers of the invention mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatin. The lubricating agent, if present, may be magnesium stearate, stearic acid or talc. If desired, the tablets may be coated with a material, such as glyceryl monostearate or glyceryl distearate, to delay absorption in the gastrointestinal tract.

Capsules for oral use include hard gelatin capsules in which the polymer is mixed with a solid diluent, and soft gelatin capsules wherein the polymer is mixed with water or an oil such as peanut oil, liquid paraffin or olive oil.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the polymer such carriers as are known in the art to be appropriate.

For parenteral use, the polymers of the present invention will generally be provided as an implant such as a stent, surgical mesh, suture or similar, such that after implantation the implant releases the active pharmaceutical ingredient(s) parenterally. Optionally however the polymers of the present invention may be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The polymers of the invention may also be presented as liposome formulations.

For topical and transdermal administration, the polymers of the invention will generally be provided in the form of ointments, cataplasms (poultices), pastes, powders, dressings, creams, films, plasters or patches.

Suitable suspensions and solutions can be used in inhalers for airway (aerosol) administration.

In one embodiment the polymers of the present invention will be administered in a form such that 0.1 to 1000 mg of H-Cap-OH or a pharmaceutically acceptable salt thereof is released into the subject per day. Preferably 1 mg to 500 mg, or 5 mg to 250 mg or 10 mg to 150 mg of H-Cap-OH or a pharmaceutically acceptable salt thereof is released into the subject per day. More preferably 15 mg to 100 mg of H-Cap-OH or a pharmaceutically acceptable salt thereof is released into the subject per day. Most preferably 20 mg to 50 mg of H-Cap-OH or a pharmaceutically acceptable salt thereof is released into the subject per day.

A tenth aspect of the present invention relates to a polymer, pharmaceutical composition or medical product of the ninth aspect of the present invention, for use in the treatment or prevention of collagen deposition, fibrosis, scars, burns, or unwanted tissue formation.

An eleventh aspect of the present invention relates to a method for the treatment or prevention of collagen deposition, fibrosis, scars, burns, or unwanted tissue formation, comprising administering to a subject in need thereof a therapeutically or prophylactically effective amount of a polymer according to any of the first, second, third or sixth aspects of the present invention, or a pharmaceutical composition according to the seventh aspect of the present invention, or a medical product according to the eighth aspect of the present invention.

A twelfth aspect of the present invention relates to a polymer comprising one or more -Cap- units of formula II:

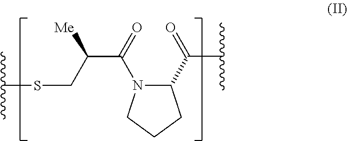

for use in the treatment or prevention of collagen deposition, fibrosis, scars, burns, or unwanted tissue formation.

A thirteenth aspect of the present invention relates to a method for the treatment or prevention of collagen deposition, fibrosis, scars, burns, or unwanted tissue formation, comprising administering to a subject in need thereof a therapeutically or prophylactically effective amount of a polymer comprising one or more -Cap- units of formula II:

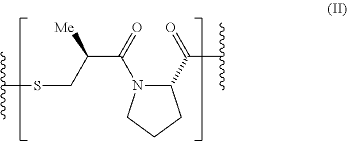

In one embodiment of the twelfth or thirteenth aspects of the present invention, the polymer comprises two or more, five or more, ten or more, or 100 or more -Cap- units.

In one embodiment of the twelfth or thirteenth aspects of the present invention, the polymer comprises one or more, two or more, five or more, ten or more, or 100 or more -Cap- units within the polymer backbone, preferably at non-terminal position(s).

Preferably the polymer comprises from 2 to 200,000-Cap- units within the polymer and/or polymer backbone. Preferably the polymer comprises from 5 to 10,000-Cap- units within the polymer and/or polymer backbone. More preferably the polymer comprises from 10 to 1,000-Cap- units within the polymer and/or polymer backbone. Optionally the polymer comprises from 20 to 100-Cap- units within the polymer and/or polymer backbone.

In another embodiment of the twelfth or thirteenth aspects of the present invention, the polymer is a copolymer, such as an alternating copolymer, a periodic copolymer, a random copolymer or a block copolymer, formed with one or more additional polymeric substances. Preferably, one or more of the additional polymeric substances is a biodegradable polymer, such as one selected from the group consisting of poly-lactic acids, poly-lactides, polylactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof. Alternatively or in addition, one or more of the additional polymeric substances may be a non-biodegradable polymer, such as one selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

In one embodiment of the twelfth or thirteenth aspects of the present invention, the polymer is appended to, terminates or cross-links the backbone(s) of one or more base polymers.

In one embodiment of the twelfth or thirteenth aspects of the present invention, the polymer is appended by or terminated with one or more groups selected from R⁹-Cap- and Cap R¹⁰, wherein R⁹ and R¹⁰ are as defined above.

In another embodiment of the twelfth or thirteenth aspects of the present invention, the polymer further comprises one or more additional active pharmaceutical ingredients appended to, terminating or incorporated in the backbone of the polymer. Preferably one or more of the additional active pharmaceutical ingredients are ACE inhibitors, such as captopril, zofenopril, zofenoprilat, lisinopril, benazepril, benazeprilat, cilazapril, cilazaprilat, moexipril, moexiprilat, perindopril, perindoprilat, quinapril, quinaprilat, ramipril, ramiprilat, spirapril, spiraprilat, trandolapril, trandolaprilat, alacepril, desacetyl-alacepril, delapril, delaprilat, imidapril, imidaprilat, rentiapril, temocapril, temocaprilat, ceronapril, enalapril, enalaprilat, moveltipril, pivalopril, despivaloyl-pivalopril, fosinopril or fosinoprilat. More preferably one or more of the additional active pharmaceutical ingredients are captopril, alacepril, rentiapril, pivalopril, despivaloyl-pivalopril, zofenopril or zofenoprilat. More preferably still one or more of the additional active pharmaceutical ingredients are captopril, zofenopril or zofenoprilat.

One or more of the additional active pharmaceutical ingredients may also be selected from the group consisting of vasopeptidase inhibitors, i.e. compounds which inhibit both angiotensin converting enzyme and neutral endopeptidase. Exemplary vasopeptidase inhibitors suitable for use in the present invention include fasidotril, fasidotrilat omapatrilat, sampatrilat, ilepatril (AVE 7688), CGS 35601, CGS 37808 and derivatives thereof. Preferably the vasopeptidase inhibitor is selected from fasidotrilat, omapatrilat, des-acetyl ilepatril, CGS 35601 and derivatives thereof.

One or more of the additional active pharmaceutical ingredients may also be selected from other inhibitors of the renin-angiotensin pathway including renin inhibitors such as aliskiren or remikiren; angiotensin II antagonists such as azilsartan, candesartan, eprosartan, irbesartan, losartan, olmesartan, tasosartan, telmisartan or valsartan; and/or an active peptide fragment of angiotensin I or II including Ang (1-7), i.e. NorLeu(1-7) or an analogue thereof such as NorLeu3-A(1-7).

One or more of the additional active pharmaceutical ingredients may also be selected from COX inhibitors including bendazac, etofenamate or fluproquazone; salicylates such as aspirin, aloxiprin, benorylate, diflunisal, ethenzamide, magnesium salicylate, methyl salicylate, salsalate, salicin, salicylamide or sodium salicylate; arylalkanoic acids such as diclofenac, aceclofenac, acemetacin, alclofenac, bromfenac, etodolac, indometacin, indometacin farnesil, nabumetone, oxametacin, proglumetacin, sulindac or tolmetin; 2-arylpropionic acids such as ibuprofen, alminoprofen, benoxaprofen, carprofen, dexibuprofen, dexketoprofen, fenbufen, felbinac, fenoprofen, flunoxaprofen, flurbiprofen, ibuproxam, indoprofen, ketoprofen, ketorolac, loxoprofen, miroprofen, naproxen, oxaprozin, pirprofen, suprofen, tarenflurbil or tiaprofenic acid; N-arylanthranilic acids such as mefenamic acid, flufenamic acid, meclofenamic acid or tolfenamic acid; pyrazolidine derivatives such as phenylbutazone, ampyrone, azapropazone, clofezone, kebuzone, metamizole, mofebutazone, oxyphenbutazone, phenazone or sulfinpyrazone; oxicams such as piroxicam, droxicam, lornoxicam, meloxicam, tenoxicam or ampiroxicam; selective COX-2 inhibitors such as celecoxib, deracoxib, etoricoxib, firocoxib, lumiracoxib, parecoxib, rofecoxib or valdecoxib; sulfonanilides such as nimesulide; and COX-inhibiting nitric oxide donators such as naproxcinod.

One or more of the additional active pharmaceutical ingredients may also be selected from glucocorticoids including non-halogenated glucocorticoids such as cortisone, hydrocortisone, budesonide, ciclesonide, cortivazol, deflazacort, hydrocortisone aceponate, hydrocortisone buteprate, hydrocortisone butyrate, meprednisone, methylprednisolone, methylprednisolone aceponate, prednicarbate, prednisolone, prednisone, prednylidene or rimexolone; halogenated glucocorticoids such as beclometasone, betamethasone, cloprednol, dexamethasone, fluticasone, flunisolide, mometasone furoate, paramethasone or triamcinolone; or selected from osteopontin inhibitors such as the antisense (AS) oligodeoxynucleotides described in J Exp Med, 2008, vol. 205(1), pp. 43-51.

In another embodiment of the twelfth or thirteenth aspects of the present invention, the polymer is biodegradable. Preferably the polymer is entirely biodegradable. Preferably the decomposition products of the polymer are only active pharmaceutical ingredients and/or pharmaceutically acceptable decomposition products.

In yet another embodiment of the twelfth or thirteenth aspects of the present invention, the polymer has an average molecular weight of from 400 to 5,000,000. Preferably the polymer has an average molecular weight of from 1,000 to 1,000,000. Most preferably the polymer has an average molecular weight of from 10,000 to 400,000.

In an alternate embodiment of the twelfth or thirteenth aspects of the present invention, the polymer has an average molecular weight of from 350 to 10,000. Preferably the polymer has an average molecular weight of from 1,200 to 2,000. Most preferably the polymer has an average molecular weight of about 1,600.

In a preferred embodiment of the twelfth or thirteenth aspects of the present invention, the polymer is one according to any of the first, second, third or sixth aspects of the present invention. In another embodiment, the polymer may be one according to the second aspect of the present invention with the exception that the unit of formula III may comprise one or more -Cap-O— units.

In one embodiment of the twelfth or thirteenth aspects of the present invention, less than 50% of the monomers of the polymer are glycolic acid. More preferably, less than 40%, less than 20%, less than 10% or less than 5% of the monomers of the polymer are glycolic acid. Most preferably none of the monomers of the polymer are glycolic acid.

In another embodiment of the twelfth or thirteenth aspects of the present invention, less than 50% of the monomers of the polymer are hydroxyalkyl acids. More preferably, less than 40%, less than 20%, less than 10% or less than 5% of the monomers of the polymer are hydroxyalkyl acids. Most preferably none of the monomers of the polymer are hydroxyalkyl acids.

In one embodiment of any of the tenth to thirteenth aspects of the present invention, the fibrosis or unwanted tissue formation is non-cancerous and/or non-vascular.

In one embodiment of any of the ninth to thirteenth aspects of the present invention, the use or method is selected from the suture of wounds such as traumatic or surgical wounds; the treatment or prevention of pathological scars, for example in situations that predispose to hypertrophy and/or in susceptible individuals such as keloid formers; plastic surgery; keyhole surgery; scar revision surgery; the reduction or prevention of vocal fold scarring preferably in order to maintain normal phonation after surgical intervention; the treatment or prevention of thermal, chemical or electrical burns; all surgery where scarring will impede the outcome from a functional and/or mechanical perspective, such as ocular and orthopaedic surgery; the treatment or prevention of adhesions, in particular those related to viscera; the treatment or prevention of pathological scars; non- or minimally-invasive procedures to reduce gastrointestinal fibrous strictures, such as those arising from Crohn's disease or oesophageal strictures, or fibrous strictures associated with other tracts such as the urethral and biliary tracts, or fibrous strictures associated with any body cavity; the treatment or prevention of gingival overgrowth, such as by the insertion of the polymer or composition as a semi-solid into the gingival pseudopocket; the treatment or prevention of excess fibrous deposition in articular, capsular, tendinous and/or ligamentous tissues; the prevention and/or reversal of fibrous compression upon neural structures such as the spinal cord and nerve tracts; the treatment or prevention of masses and tumours that consist in part or whole of fibrous tissue, in which the reduction of fibrous material is beneficial; the treatment or prevention of leiomyomata; the treatment or prevention of fibrous proliferative disorders such as those of the skin or other external or internal body surfaces, including digital fibroma; the treatment or prevention of deformities, fusion of digits and contractures associated with abnormal scarring in epidermolysis bullosa; the treatment or prevention of ulcers such as mouth ulcers; the treatment or prevention of gastrointestinal strictures that are common during disease flare-up in Crohn's disease; the treatment or prevention of cutaneous sulphur mustard injuries, such as those arising from chemical warfare; the reduction of excessive angiogenesis and/or vascular angiogenesis; the treatment or prevention of restenosis; the maintenance of the operation of an implanted medical device preferably by the prevention of obstruction such as the maintenance of indwelling catheter/cannula patency, the maintenance of indwelling catheter/cannula access, the maintenance of pressure monitoring device patency, the maintenance of prosthetic valve implant patency, the maintenance of shunt e.g. neural, central nervous system, cardiovascular, gastrointestinal or biliary shunt patency, the maintenance of ocular drainage device patency, the maintenance of surgical wound drainage patency, the maintenance of patency of intraocular lens, the maintenance of patency of a penile implant, the maintenance of intravascular implant patency, the maintenance of patency of implant for haemodialysis access or the maintenance of patency of a peritoneal dialysis catheter implant; the treatment or prevention of foreign body granuloma of skin; the treatment or prevention of fibroproliferative polyps; the treatment or prevention of fibroproliferative disorders of the skin, lung, liver, kidney, peripheral- or cardio-vasculature; the treatment or prevention of capsule formation around silicone breast implants and other silicone implants; the treatment or prevention of sclerosing lobular hyperplasia of the breast after reduction mammaplasty; the creation or restoration of fallopian tube patency, optionally in conjunction with fimbrioplasty and/or tuboplasty; the treatment or prevention of caesarean scarring; the prevention or reduction in scarring at donor and recipient sites of hair transplantation; and the prevention or reduction of scarring associated with ethmoid sinus surgery.

A fourteenth aspect of the present invention relates to a polymer, pharmaceutical composition, medical product, process or method according to any of the preceding aspects of the present invention, wherein each -Cap- unit is instead independently defined as any unit such that H-Cap-OH or a pharmaceutically acceptable salt or derivative thereof is capable of inhibiting an angiotensin converting enzyme.

In one embodiment of the fourteenth aspect of the present invention, each -Cap- unit is the same, i.e. the polymer contains only one type of -Cap- unit. Alternately the polymer used in, or of the fourteenth aspect of the present invention may contain a plurality of structurally different types of -Cap- unit. Preferably the polymer used in, or of, the fourteenth aspect of the present invention contains no more than 5 structurally different types of -Cap- unit. More preferably it contains no more than 3 structurally different types of -Cap- unit. Most preferably it contains no more than 2 structurally different types of -Cap- unit.

Where a polymer used in, or of, the fourteenth aspect of the present invention comprises two or more structurally different types of -Cap- unit, one of said structurally different types of -Cap- unit may optionally be selected from a unit of formula II as defined in accordance with the first aspect of the present invention.

In one embodiment of the fourteenth aspect of the present invention, at least one type of -Cap- unit is defined such that H-Cap-OH or a pharmaceutically acceptable salt or derivative thereof is capable of inhibiting an angiotensin-I converting enzyme. Preferably each structurally different type of -Cap- unit is defined such that H-Cap-OH or a pharmaceutically acceptable salt or derivative thereof is capable of inhibiting an angiotensin-I converting enzyme.

In another embodiment of the fourteenth aspect of the present invention, at least one type of -Cap- unit is defined such that H-Cap-OH or a pharmaceutically acceptable salt or derivative thereof is capable of inhibiting vasopeptidase. Optionally each structurally different type of -Cap- unit is defined such that H-Cap-OH or a pharmaceutically acceptable salt or derivative thereof is capable of inhibiting vasopeptidase. For instance, H-Cap-OH may be selected from fasidotrilat, omapatrilat, des-acetyl ilepatril and CGS 35601.

In one embodiment of the fourteenth aspect of the present invention, at least one type of -Cap- unit is defined such that H-Cap-OH comprises a thiol group and a carboxylic acid group. Preferably each structurally different type of -Cap- unit is defined such that H-Cap-OH comprises a thiol group and a carboxylic acid group.

In another embodiment of the fourteenth aspect of the present invention, at least one type of -Cap- unit is defined as a unit of formula IIa:

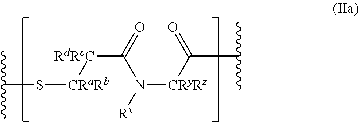

wherein R^{a}, R^{b}, R^{c}, R^{d}, R^{x}, R^{y} and R^{z} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y} and R^{z} together with the atom or atoms to which they are attached may form a cyclic hydrocarbyl group which may optionally be substituted and which may optionally include one or more heteroatoms N, O or S in its carbon skeleton.

Preferably R^{a}, R^{b}, R^{c} and R^{d} are each independently hydrogen or contain from 1 to 12 carbon atoms. More preferably R^{a}, R^{b}, R^{c} and R^{d} are each independently hydrogen or contain from 1 to 6 carbon atoms. Even more preferably R^{a}, R^{b} and R^{c} are hydrogen and R^{d} contains from 1 to 6 carbon atoms. Preferably R^{d} is an alkyl group. Most preferably R^{a}, R^{b} and R^{c} are hydrogen and R^{d} is methyl.

Optionally R^{x} and R^{y} together with the atoms to which they are attached form a cyclic hydrocarbyl group which may optionally be substituted and which may optionally include one or more additional heteroatoms N, O or S in its carbon skeleton. Preferably the cyclic hydrocarbyl group is a four, five or six membered ring. Most preferably the cyclic hydrocarbyl group is a five membered ring. The cyclic hydrocarbyl group may be aromatic or non-aromatic. Preferably the cyclic hydrocarbyl group is non-aromatic.

Alternatively R^{x} and R^{y} are each independently hydrogen or contain from 1 to 12 carbon atoms. More preferably R^{x} and R^{y} are each independently hydrogen or contain from 1 to 6 carbon atoms. Even more preferably R^{y} is hydrogen and R^{x} contains from 1 to 6 carbon atoms. Preferably R^{x} is an alkyl group. Most preferably R^{y} is hydrogen and R^{x} is cyclopentyl.

Preferably R^{z} is hydrogen.

Preferably each structurally different type of -Cap- unit is defined as a unit of formula IIa.

In another embodiment of the fourteenth aspect of the present invention, at least one type of -Cap- unit is defined as a unit of formula IIb:

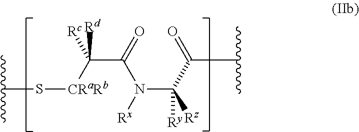

wherein R^{a}, R^{b}, R^{c}, R^{d}, R^{x}, R^{y} and R^{z} are as defined above.

Preferably each structurally different type of -Cap- unit is defined as a unit of formula IIb.

In another embodiment of the fourteenth aspect of the present invention, at least one type of -Cap- unit is defined as a unit of formula IIc:

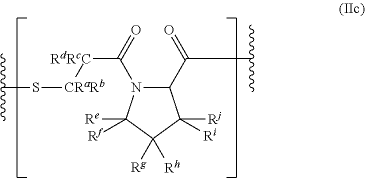

wherein W, R^{b}, R^{c} and R^{d} are as defined above;
R^{e}, R^{f}, R^{g}, R^{h}, Rⁱ and R^{j} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton;
and wherein any two or more of R^{a}, R^{b}, R^{c}, R^{d}, R^{e}, R^{f}, R^{g}, R^{h}, Rⁱ and R^{j} together with the atom or atoms to which they are attached may form a cyclic hydrocarbyl group which may optionally be substituted and which may optionally include one or more heteroatoms N, O or S in its carbon skeleton.

Preferably R^{e}, R^{f}, R^{g}, R^{h}, Rⁱ and R^{j} are each independently hydrogen or contain from 1 to 12 carbon atoms. More preferably R^{e}, R^{f}, R^{g}, R^{h}, Rⁱ and R^{j} are each independently hydrogen or contain from 1 to 6 carbon atoms. Even more preferably R^{e}, R^{f}, R^{h}, Rⁱ and R^{j} are hydrogen and R^{g} is hydrogen or a —S-alkyl, —S-alkenyl, —S-alkynyl, —S-aryl, —S-arylalkyl, —S-arylalkenyl, —S-arylalkynyl, —S-alkylaryl, —S-alkenylaryl or —S-alkynylaryl group. Most preferably R^{e}, R^{f}, R^{h}, Rⁱ and R^{j} are hydrogen and R^{g} is hydrogen or —SPh.

Preferably each structurally different type of -Cap- unit is defined as a unit of formula IIc.

In yet another embodiment of the fourteenth aspect of the present invention, at least one type of -Cap- unit is defined as a unit of formula IId:

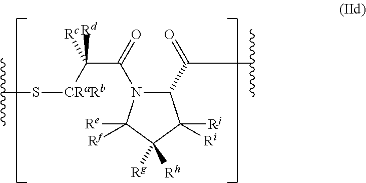

wherein R^{a}, R^{b}, R^{c}, R^{d}, R^{e}, R^{f}, R^{g}, R^{h}, Rⁱ and R^{j} are as defined above.

Preferably each structurally different type of -Cap- unit is defined as a unit of formula IId.

In a preferred embodiment of the fourteenth aspect of the present invention, at least one type of -Cap- unit is defined as a unit of formula IIe:

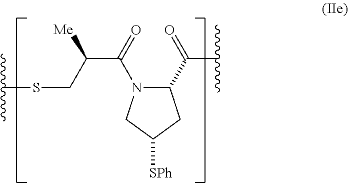

Thus, it can be seen that where -Cap- is a unit of formula IIe, H-Cap-OH represents the active metabolite zofenoprilat of the ACE inhibitor zofenopril, PhCO-Cap-OH.

More preferably each -Cap- unit is defined as a unit of formula IIe.

In another embodiment of the fourteenth aspect of the present invention, at least one type of -Cap- unit is defined as a unit of formula

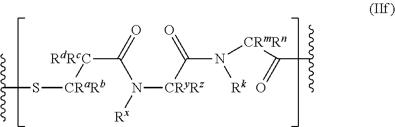

wherein R^{a}, R^{b}, R^{c}, R^{d}, R^{x}, R^{y} and R^{z} are as defined above;
R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton;
and wherein any two or more of R^{a}, R^{b}, R^{c}, R^{d}, R^{x}, R^{y}, R^{z}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton.

Preferably R^{k} and R^{m} are each independently hydrogen or contain from 1 to 12 carbon atoms. More preferably R^{k} and R^{m} are each independently hydrogen or contain from 1 to 6 carbon atoms. Most preferably R^{k} and R^{m} are hydrogen.

Preferably Rⁿ is hydrogen or contains from 1 to 12 carbon atoms. More preferably Rⁿ is an arylalkyl group which may optionally be substituted and which may optionally include one or more heteroatoms N, O or S in its carbon skeleton. More preferably still Rⁿ is an unsubstituted arylalkyl group containing from 7 to 12 carbon atoms. Most preferably Rⁿ is —CH₂Ph.

Preferably each structurally different type of -Cap- unit is defined as a unit of formula IIf.

In another embodiment of the fourteenth aspect of the present invention, at least one type of -Cap- unit is defined as a unit of formula IIg:

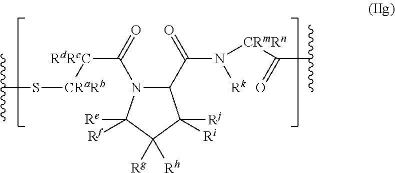

wherein R^{a}, R^{b}, R^{c}, R^{d}, R^{e}, R^{f}, R^{g}, R^{h}, Rⁱ, R^{j}, R^{k}, R^{m} and Rⁿ are as defined above;
and wherein any two or more of R^{a}, R^{b}, R^{c}, R^{d}, R^{e}, R^{f}, R^{g}, R^{h}, Rⁱ, R^{j}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton.

Preferably each structurally different type of -Cap- unit is defined as a unit of formula IIg.

In another embodiment of the fourteenth aspect of the present invention, at least one type of -Cap- unit is defined as a unit of formula IIh:

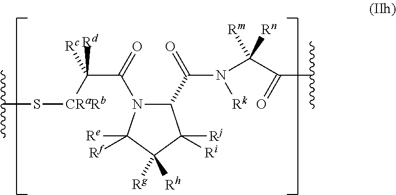

wherein R^{a}, R^{b}, R^{c}, R^{d}, R^{e}, R^{f}, R^{g}, R^{h}, Rⁱ, R^{j}, R^{k}, R^{m} and Rⁿ are as defined above.

Preferably each structurally different type of -Cap- unit is defined as a unit of formula IIh.

In a preferred embodiment of the fourteenth aspect of the present invention, at least one type of -Cap- unit is defined as a unit of formula IIi:

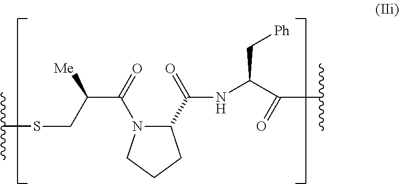

Thus, it can be seen that where -Cap- is a unit of formula IIi, H-Cap-OH represents the active metabolite desacetyl-alacepril of the ACE inhibitor alacepril, Ac-Cap-OH.

More preferably each -Cap- unit is defined as a unit of formula IIi.

In another embodiment of the fourteenth aspect of the present invention, at least one type of -Cap- unit is defined as a unit of formula IIj:

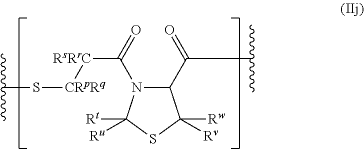

wherein R^{P}, R^{q}, R^{r}, R^{s}, R^{t}, R^{u}, R^{v} and R^{w} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{P}, R^{q}, R^{r}, R^{x}, R^{t}, R^{v} and R^{w} together with the atom or atoms to which they are attached may form a cyclic hydrocarbyl group which may optionally be substituted and which may optionally include one or more heteroatoms N, O or S in its carbon skeleton.

Preferably R^{P}, R^{q}, R^{r} and R^{s} are each independently hydrogen or contain from 1 to 12 carbon atoms. More preferably R^{P}, R^{q}, R^{r} and R^{s} are each independently hydrogen or contain from 1 to 6 carbon atoms. Even more preferably R^{P}, R^{q}, R^{r} and R^{s} are hydrogen.

Preferably R^{t}, R^{u}, R^{v} and R^{w} are each independently hydrogen or contain from 1 to 12 carbon atoms. More preferably R^{t}, R^{u}, R^{v} and R^{w} are each independently hydrogen or contain from 1 to 6 carbon atoms. Even more preferably R^{u}, R^{v} and R^{w} are hydrogen and R^{t} is a substituted or unsubstituted aryl, alkylaryl, alkenylaryl or alkynylaryl group. More preferably still R^{u}, R^{v} and R^{w} are hydrogen and R^{t} is a substituted aryl group, preferably substituted with one or more —OH groups. Most preferably R^{u}, R^{v} and R^{w} are hydrogen and R^{t} is an ortho-hydroxyphenyl group.

Preferably each structurally different type of -Cap- unit is defined as a unit of formula IIj.

In another embodiment of the fourteenth aspect of the present invention, at least one type of -Cap- unit is defined as a unit of formula IIk:

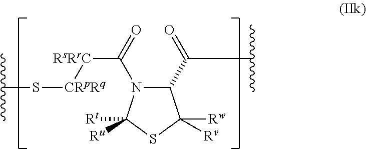

wherein R^{P}, R^{q}, R^{r}, R^{s}, R^{t}, R^{u}, R^{v} and R^{w} are as defined above.

Preferably each structurally different type of -Cap- unit is defined as a unit of formula IIk.

In a preferred embodiment of the fourteenth aspect of the present invention, at least one type of -Cap- unit is defined as a unit of formula IIm:

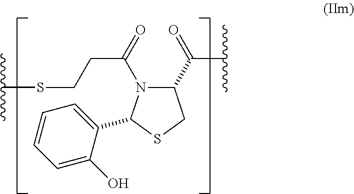

Thus, it can be seen that where -Cap- is a unit of formula IIm, H-Cap-OH represents the ACE inhibitor rentiapril.

More preferably each -Cap- unit is defined as a unit of formula IIm.

In another preferred embodiment of the fourteenth aspect of the present invention, at least one type of -Cap- unit is defined as a unit of formula IIn:

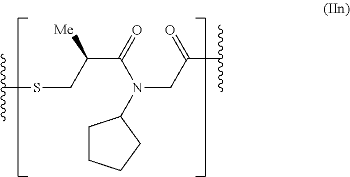

Thus, it can be seen that where -Cap- is a unit of formula IIn, H-Cap-OH represents the active metabolite despivaloyl-pivalopril of the ACE inhibitor pivalopril, (CH₃)₃CCO-Cap-OH.

More preferably each -Cap- unit is defined as a unit of formula IIn.

For the avoidance of doubt, in relation to all aspects of the present invention it is noted that the symbol “-Cap-” is orientation specific. In other words, where the unit -Cap- is defined in accordance with a chemical structure as illustrated herein, the “—C” terminal of -Cap- is representative of the terminal bond illustrated by the wavey line on the left hand side of the structure, whereas the “p-” terminal is representative of the terminal bond on the right hand side of the structure.

It therefore follows that unless the chemical structure of -Cap- is symmetrical, where A≠B, A-Cap-B≠B-Cap-A. Accordingly, where -Cap- is a unit of formula II, A-Cap-B is representative only of:

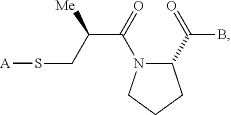

whereas B-Cap-A is representative only of:

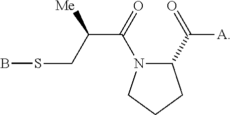

Similarly, in relation to all aspects of the present invention it is noted that the symbols “-L-” and “-L′-” are orientation specific. In other words, where a unit -L- or -L′- is defined in accordance with a chemical structure as illustrated herein, the “-L” or “-L′” terminal of -L- or -L′- is representative of the terminal bond illustrated by the wavey line on the left hand side of the structure, whereas the “L-” or “L′-” terminal is representative of the terminal bond on the right hand side of the structure.

It therefore follows that unless the chemical structure of -L- or -L′- is symmetrical, where R¹≠R², R¹-L-R²≠R²-L-R¹ and R¹-L′-R²≠R²-L′-R¹. Thus for example, where -L- is a unit of formula IIIb, R¹-L-R² is representative only of:

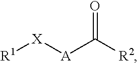

whereas R²-L-R¹ is representative only of:

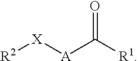

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### DEFINITIONS

As used herein, the term “polymer” refers to any compound comprising one or more types of repeating structural unit and includes pharmaceutically acceptable salts of said polymer. Each of said repeating structural units is referred to as a “monomer unit”. The monomer units may or may not be directly bonded to each other (i.e. linked by a covalent bond with no intervening atoms or groups present). The polymers according to the invention include cyclic, branched and linear polymers. Preferably the polymers according to the invention are branched or linear. Most preferably the polymers according to the invention are linear.

In one embodiment, the polymers according to any aspect of the present invention do not comprise Au(I). Preferably, the polymers do not comprise any Au atoms. More preferably the polymers do not comprise any Cu, Ag or Au atoms. More preferably still the polymers do not comprise any metal atoms.

As used herein, the term “homo-polymer” refers to any polymer that comprises only one type of repeating structural unit, such as a repeating -Cap- unit of formula II.

As used herein, the term “copolymer” refers to any polymer comprising two or more different types of repeating structural unit and includes terpolymers and the like and also pharmaceutically acceptable salts of said polymer. For the avoidance of doubt, where it is stated that a copolymer is formed between a first type of repeating structural unit and one or more “additional polymeric substances”, this means that the copolymer incorporates one or more monomer units from the additional polymeric substance(s).

The term “copolymer” includes “alternating copolymers”, i.e. those in which the different types of monomer unit alternate along the polymer chain, e.g. -A-B-A-B-A-B-; “periodic copolymers”, i.e. those in which the different types of monomer unit are arranged in a repeating sequence, e.g. -(A-B-B-A-B-A-A-A)ₙ-; “random copolymers”, i.e. those with random sequences of the different types of monomer unit; and “block copolymers”, i.e. those which comprise two or more homo-polymer subunits linked by one or more covalent bonds, e.g. -A-A-A-A-A-B-B-B-B-B.

As used herein, the “backbone” of the polymer refers to the linear chain of atoms within the polymer to which all other chains, long or short or both, may be regarded as being pendant. Where two or more chains could equally be considered to be the backbone, that one is selected which leads to the simplest representation of the polymer.

Where a group is “appended to” the backbone of a polymer this refers to a group that is not itself part of the backbone of said polymer, but that is covalently bound to a non-terminal monomer unit within the backbone of said polymer. Where a group “terminates” the backbone of a polymer this refers to a group that is covalently bound to an end or terminal monomer unit of the backbone of said polymer. As used herein, a unit or group such as a monomer unit at a “terminal position” refers to a unit or group covalently attached to the polymer backbone that does not covalently link two or more other monomer units.

A “cross-linking group” refers to a group that cross-links the backbone(s) of one or more polymers such as base polymers. Where a group “cross-links” the backbone(s) of one or more polymer(s), this refers to a group that is covalently bound to two or more monomer units either within the same polymer backbone such that the polymer backbone is cross-linked or within different polymer backbones such that two or more polymer backbones are joined together. Preferably where a group “cross-links” the backbone(s) of one or more polymer(s), this refers to a group that is covalently bound to two or more monomer units within different polymer backbones. Preferably the two or more monomer units are not at a terminal position of the polymer backbone(s).

Where any group is covalently bound to a monomer unit, the individual monomer unit in question may be chemically modified, optionally with the inclusion of a linker atom or group, to allow for such bonding.

As used herein, the term “molecular weight” refers to molecular weight as measured in Daltons (Da).

As used herein, a “biodegradable” polymer refers to a polymer that undergoes hydrolysis on contact with biological fluids or systems such as blood plasma, skin or gastric fluid. Conversely a “non-biodegradable” polymer refers to a polymer that does not undergo hydrolysis on contact with biological fluids or systems. A polymer that is “entirely biodegradable” refers to a polymer wherein at least one covalent bond in every link between constituent monomer units is able to undergo hydrolysis on contact with biological fluids or systems.

In one embodiment of any aspect of the invention, a “biodegradable” polymer undergoes hydrolysis on contact with an aqueous solution of pH between 5 and 9, preferably between 6 and 8, more preferably about 7.

In another embodiment of any aspect of the invention, a “biodegradable” polymer undergoes hydrolysis on contact with an aqueous solution of pH less than 5, less than 4, less than 3, less than 2 or preferably of pH about 1.2.

Preferably a “biodegradable” polymer undergoes hydrolysis on contact with biological fluids or systems at a rate such that it takes on average at least 10 days for the polymer to degrade into its constituent monomer units and/or constituent non-biodegradable sections. More preferably it takes on average at least 20 days, at least 30 days or at least 40 days for the polymer to degrade into its constituent monomer units and/or constituent non-biodegradable sections. Most preferably it takes on average at least 50 days for the polymer to degrade into its constituent monomer units and/or constituent non-biodegradable sections.

Preferably a “biodegradable” polymer undergoes hydrolysis on contact with biological fluids or systems at a rate such that it takes on average less than 400 days for the polymer to degrade into its constituent monomer units and/or constituent non-biodegradable sections. More preferably it takes on average less than 200 days for the polymer to degrade into its constituent monomer units and/or constituent non-biodegradable sections. Most preferably it takes on average less than 100 days for the polymer to degrade into its constituent monomer units and/or constituent non-biodegradable sections.

Where a first bond is said to be more resistant to hydrolysis than a second bond, it is to be understood that said first bond hydrolyses at a slower rate than the second bond on contact with biological fluids or systems. In one embodiment this rate is measured in an aqueous solution of pH between 5 and 9, preferably between 6 and 8, more preferably about 7. In another embodiment this rate is measured in an aqueous solution of pH less than 5, less than 4, less than 3, less than 2 or preferably of pH about 1.2.

As used herein, the term “pharmaceutically acceptable decomposition products” refers to any decomposition products which do not have unacceptable adverse effects on a subject (e.g. human or other animal) to be treated, and may include for example pharmaceutically acceptable excipients. Preferably the pharmaceutically acceptable decomposition products have no adverse effects, and even more preferably they are inactive, that is to say they have no discernible biological effects on the subject.

Active pharmaceutical ingredients and pharmaceutically acceptable excipients are well known to those skilled in the art and can be found for instance by reference to The Merck Index, 14^{th} Ed. 2006, or pharmacopoeias such as the British Pharmacopoeia 2009 or the European Pharmacopoeia, 6^{th} Ed. 2007.

As used herein, a “base polymer” can be any polymer to which one or more additional groups are covalently bound, but is preferably one that does not comprise a -Cap- unit. Preferably any polymer comprising one or more base polymers comprises at least 50 wt. % of the base polymer(s). Optionally, any polymer comprising one or more base polymers comprises at least 75 wt. % of the base polymer(s).

As used herein, the “chain length” of a linker L refers to the number of chemical bonds between the two bonding termini of said linker, as measured by the most direct route.

As used herein, a “protected derivative” of a compound refers to a compound in which one or more chemically active groups, such as hydroxyl groups, thiol groups, amines, carbonyl groups, carboxyl groups and the like are protected with one or more protecting groups. Suitable protecting groups for protecting chemically active groups are known in the art, for example from “Protective Groups in Organic Synthesis” by T. W. Greene and P. G. M. Wuts (Wiley-Interscience, 4^{th} Ed. 2006).

For example, a protected derivative of H-Cap-OH may include compounds in which the thiol group is protected such as PhCH₂-Cap-OH, 4-MeOC₆H₄CH₂-Cap-OH, 4-NO₂C₆H₄CH₂-Cap-OH, Ph₂CH-Cap-OH, EtNHCO-Cap-OH and pharmaceutically acceptable salts thereof, and/or compounds in which the carboxyl group is protected such as H-Cap-OMe, H-Cap-O′Bu, H-Cap-OCH₂Ph and pharmaceutically acceptable salts thereof.

As used herein, “chemical activation” of the —COOH group refers to the use of chemical reagents to convert the —COOH group into a species that is more reactive towards nucleophilic attack, for example by thiols or thiolate anions. Methods of performing such chemical activation are well known to the person skilled in the art and include for instance the conversion of the —COOH group into an acyl halide such as —COCl, into an anhydride such as —C(O)OC(O)OMe, or into an active ester such as a pentafluorophenyl ester (—COOPfp), or the use of coupling reagents such as DCCI (dicyclohexylcarbodiimide) and

HOBt (1-hydroxybenzotriazole), TBTU (O-(benzotriazol-1-yl)-N,N,N′,N′-tetramethyl-uronium tetrafluoroborate or the guanidinium N-oxide isomer thereof) or HATU (O-(7-azabenzotriazol-1-yl)-N,N,N′,N′-tetramethyluronium hexafluorophosphate or the guanidinium N-oxide isomer thereof).

As used herein, the act of “administering” a medical product is to be understood as including any act wherein the medical product is placed in contact with the subject, such as the acts of inserting or implanting the medical product in a subject where for example the medical product is a stent, suture or catheter, or the act of applying the medical product to the external surface of the subject where for example the medical product is a plaster or cling-film.

As used herein, a “minimally invasive” procedure refers to any procedure that is less invasive than the equivalent open-surgery performed for the same purpose.

For the purposes of the present invention, an “alkyl” group is defined as a monovalent saturated hydrocarbon, which may be straight-chained or branched, or be or include cyclic groups. An alkyl group may optionally include one or more heteroatoms N, O or S in its carbon skeleton. Examples of alkyl groups are methyl, ethyl, n-propyl, i-propyl, n-butyl, butyl, t-butyl and n-pentyl groups. Preferably an alkyl group is straight-chained or branched and does not include any heteroatoms in its carbon skeleton. Preferably an alkyl group is a C₁-C₁₂ alkyl group, which is defined as an alkyl group containing from 1 to 12 carbon atoms. More preferably an alkyl group is a C₁-C₆ alkyl group, which is defined as an alkyl group containing from 1 to 6 carbon atoms. An “alkylene” group is similarly defined as a divalent alkyl group.

An “alkenyl” group is defined as a monovalent hydrocarbon, which comprises at least one carbon-carbon double bond, which may be straight-chained or branched, or be or include cyclic groups. An alkenyl group may optionally include one or more heteroatoms N, O or S in its carbon skeleton. Examples of alkenyl groups are vinyl, allyl, but-1-enyl and but-2-enyl groups. Preferably an alkenyl group is straight-chained or branched and does not include any heteroatoms in its carbon skeleton. Preferably an alkenyl group is a C₂-C₁₂ alkenyl group, which is defined as an alkenyl group containing from 2 to 12 carbon atoms. More preferably an alkenyl group is a C₂-C₆ alkenyl group, which is defined as an alkenyl group containing from 2 to 6 carbon atoms. An “alkenylene” group is similarly defined as a divalent alkenyl group.

An “alkynyl” group is defined as a monovalent hydrocarbon, which comprises at least one carbon-carbon triple bond, which may be straight-chained or branched, or be or include cyclic groups. An alkynyl group may optionally include one or more heteroatoms N, O or S in its carbon skeleton. Examples of alkynyl groups are ethynyl, propargyl, but-1-ynyl and but-2-ynyl groups. Preferably an alkynyl group is straight-chained or branched and does not include any heteroatoms in its carbon skeleton. Preferably an alkynyl group is a C₂-C₁₂ alkynyl group, which is defined as an alkynyl group containing from 2 to 12 carbon atoms. More preferably an alkynyl group is a C₂-C₆ alkynyl group, which is defined as an alkynyl group containing from 2 to 6 carbon atoms. An “alkynylene” group is similarly defined as a divalent alkynyl group.

An “aryl” group is defined as a monovalent aromatic hydrocarbon. An aryl group may optionally include one or more heteroatoms N, O or S in its carbon skeleton. Examples of aryl groups are phenyl, naphthyl, anthracenyl and phenanthrenyl groups. Preferably an aryl group does not include any heteroatoms in its carbon skeleton. Preferably an aryl group is a C₄-C₁₄ aryl group, which is defined as an aryl group containing from 4 to 14 carbon atoms. More preferably an aryl group is a C₆-C₁₀ aryl group, which is defined as an aryl group containing from 6 to 10 carbon atoms. An “arylene” group is similarly defined as a divalent aryl group.

An “acyl” group is defined as any —CO-alkyl, —CO-alkenyl, —CO-alkynyl, —CO-aryl, —CO-arylalkyl, —CO-arylalkenyl, —CO-arylalkynyl, —CO-alkylaryl, —CO-alkenylaryl or —CO-alkynylaryl group. Accordingly an acyl group can alternatively be seen as an alkyl, alkenyl, alkynyl, arylalkyl, arylalkenyl or arylalkynyl group substituted with a ═O substituent on the same atom by which the moiety is attached to the rest of the molecule. An acyl group may optionally include one or more heteroatoms N, O or S in its carbon skeleton. Examples of acyl groups are acetyl, benzoyl and acryloyl groups. Preferably an acyl group does not include in its carbon skeleton a heteroatom directly bonded to the —CO— group by which the moiety is attached to the rest of the molecule. Preferably an acyl group is straight-chained or branched and does not include any heteroatoms in its carbon skeleton.

Preferably an acyl group is a C₂-C₁₂ acyl group, which is defined as an acyl group containing from 2 to 12 carbon atoms. More preferably an acyl group is a C₂-C₆ acyl group, which is defined as an acyl group containing from 2 to 6 carbon atoms.

A “hydrocarbyl” group is defined as any monovalent group comprising hydrogen and carbon. A hydrocarbyl group may optionally include one or more heteroatoms N, O or S in its carbon skeleton. Preferably a hydrocarbyl group includes no more than one heteroatom in its carbon skeleton. More preferably a hydrocarbyl group does not include any heteroatoms in its carbon skeleton. Examples of hydrocarbyl groups include any of the alkyl, alkenyl, alkynyl or aryl groups mentioned above. Preferred hydrocarbyl groups are C₁-C₁₄ hydrocarbyl groups, more preferably C₁-C₈ hydrocarbyl groups.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

For the purposes of this invention, an optionally substituted alkyl, alkenyl, alkynyl, aryl, arylalkyl, arylalkenyl, arylalkynyl, alkylaryl, alkenylaryl, alkynylaryl or hydrocarbyl group may be substituted with one or more of —F, —Cl, —Br, —I, —CF₃, —CCl₃, —CBr₃, —CI₃, —OH, —SH, —NH₂, —CN, —NO₂, —COOH, —R^{α}—O—R^{β}, R^{α}—S—R^{β}, —R^{α}—SO—R^{β}, —R^{α}—SO₂—R^{β}, —R^{α}—SO₂—OR^{β}, —R^{α}O—SO₂—R^{β}, —R^{α}—SO₂—N(R^{β})₂, —R^{α}—NR^{β}—SO₂—R^{β}, —R^{α}O—SO₂—OR^{β}, —R^{α}O—SO₂—N(R^{β})₂, —R^{α}—NR^{β}—SO₂—OR^{β}, —R^{α}—NR^{β}—SO₂—N(R^{β})₂, —R^{α}—N(R^{β})₂, —R^{α}—N(R^{β})₃⁺, —R^{α}—P(R^{β})₂, —R^{α}—Si(R^{β})₃, —R^{α}CO—R^{β}, —R^{α}—CO—OR^{β}, —R^{α}O—CO—R^{β}, —R^{α}—CO—N(R^{β})₂, —R^{α}—NR^{β}—CO—R^{β}, —R^{α}O—CO—R^{β}, —R^{α}—CO—N(R^{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{α}O—CS—R^{β}, —R^{α}—CS—N(R^{β})₂, —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}, a bridging substituent such as —O—, —S—, —NR^{β}— or —R^{α}—, or a π-bonded substituent such as ═O, ═S or ═NR^{β}. In this context, —R^{α}— is independently a chemical bond, a C₁-C₁₀ alkylene, C₁-C₁₀ alkenylene or C₁-C₁₀ alkynylene group. —R^{β} is independently hydrogen, unsubstituted C₁-C₆ alkyl or unsubstituted C₆-C₁₀ aryl. Optional substituent(s) are preferably taken into account when calculating the total number of carbon atoms in the parent group substituted with the optional substituent(s). Preferably an optionally substituted alkyl, alkenyl, alkynyl, aryl, arylalkyl, arylalkenyl, arylalkynyl, alkylaryl, alkenylaryl, alkynylaryl or hydrocarbyl group is not substituted with a bridging substituent. Preferably an optionally substituted alkyl, alkenyl, alkynyl, aryl, arylalkyl, arylalkenyl, arylalkynyl, alkylaryl, alkenylaryl, alkynylaryl or hydrocarbyl group is not substituted with a π-bonded substituent. Preferably a substituted group comprises 1, 2 or 3 substituents, more preferably 1 or 2 substituents, and even more preferably 1 substituent.

Any optional substituent may be protected. Suitable protecting groups for protecting optional substituents are known in the art, for example from “Protective Groups in Organic Synthesis” by T. W. Greene and P. G. M. Wuts (Wiley-Interscience, 4^{th} Ed. 2006).

The compounds and polymers of the present invention can be used both, in their free base form and their acid addition salt form. For the purposes of this invention, a “salt” of a compound or polymer of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulphuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulphonic acids (for example, methanesulphonic, trifluoromethanesulphonic, ethanesulphonic, 2-hydroxyethanesulphonic, benzenesulphonic, toluene-p-sulphonic, naphthalene-2-sulphonic or camphorsulphonic acid) or amino acids (for example, ornithinic, glutamic or aspartic acid). The acid addition salt may be a mono-, di-, tri- or multi-acid addition salt. A preferred salt is a hydrohalogenic, sulphuric, phosphoric or organic acid addition salt. A more preferred salt is a hydrochloric acid addition salt.

In addition to pharmaceutically acceptable acid addition salts, other acid addition salts are included in the present invention, since they have potential to serve as intermediates in the purification or preparation of other, for example, pharmaceutically acceptable, acid addition salts, or are useful for identification, characterisation or purification of the free base.

The compounds and polymers of the present invention can also be used both, in their free acid form and their salt form. For the purposes of this invention, a “salt” of a compound or polymer of the present invention includes one formed between a carboxylic acid functionality of a compound or polymer of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt. Preferably the salt is a pharmaceutically acceptable salt.

For the avoidance of doubt, the present invention encompasses pharmaceutically acceptable salts, derivatives, solvates, clathrates and/or hydrates (including anhydrous forms) of the compounds and polymers of the present invention.

The invention will now be described with reference to the following examples. It will be appreciated that what follows is by way of example only and that modifications to detail may be made whilst still falling within the scope of the invention.

### EXAMPLES

### Example 1

### Polymerisation of Captopril

4 g of captopril in 15 ml of toluene with 0.5 g of Novozyme lipase B from Candida antartica was stirred in a 100 ml round bottom flask fitted with a drying tube (containing 3 Å molecular sieves and indicator silica gel) for 48 hours at 110° C. using a hotplate and magnetic stirrer. 50 ml of THF was then added and the enzyme was removed by filtration. The solvent was removed using rotary evaporation to yield a dark yellow viscous liquid which turned solid after 48 hours. This was recrystallised from and washed with methanol to yield a white solid insoluble in methanol and THF but soluble in chloroform.

The methanol filtrate was stored in a stoppered 100 ml round bottom flask. After 1 week it was observed that a small amount of further precipitate had formed which was then separated by decanting the supernatant and drying the white methanol-insoluble solid at room temperature on a watch glass.

Both captopril and captopril lactone are freely soluble in methanol, THF and chloroform indicating that the product is not monomeric captopril or captopril lactone.

GPC analysis of the crude mixture in THF indicated a very high molecular weight component using the right angle light scattering (RALS) detector. GPC of the purified solid in chloroform suggested the formation of dimeric captopril.

¹H NMR analysis of the purified solid in CDCl₃ indicated that there was no COOH peak but still some SH present. The spectrum was similar but distinct from that of captopril, indicating chemical modification with preservation of the core captopril structure.

IR analysis indicated a much reduced SH peak (2564 cm⁻¹) versus captopril and a shift in CO═O from 1744 cm⁻¹ to 1730 cm⁻¹ and from 1600 cm⁻¹ to 1584 cm⁻¹. Peaks observed in the captopril spectrum at 1376 cm⁻¹ and 1327 cm⁻¹, assigned to the C—O of the acid, were not present.

DSC analysis gave no Tₘ or S_{olidify}.

UV analysis showed a λₘₐₓ of 245 nm which is comparable to captopril at 244 nm. However the UV absorption peak was much broader and similar to that obtained from other polymers.

The above data is consistent with the formation of at least a dimeric captopril species linked via a thioester bond. It thus demonstrates the utility of the above method in the formation of thioester linkages between H-Cap-OH monomer units.

### Example 2

### Polymerisation of Captopril Lactone

4 g of captopril lactone in 15 ml of toluene with 0.5 g of Novozyme lipase B from Candida antartica was stirred in a 100 ml round bottom flask fitted with a drying tube (containing 3 Å molecular sieves and indicator silica gel) for 48 hours at 110° C. using a hotplate and magnetic stirrer. 50 ml of THF was then added and the enzyme was removed by filtration. The solvent was removed using rotary evaporation to yield an off-white solid. Recrystallisation from methanol produced a white solid insoluble in methanol and THF but soluble in chloroform.

The methanol filtrate was stored in a stoppered 100 ml round bottom flask. After 1 week further material had precipitated from the methanol, which was separated by decanting the supernatant and drying the white methanol-insoluble solid at room temperature on a watch glass.

The combined yield was about 2-2.5 g.

Both captopril and captopril lactone are freely soluble in methanol, THF and chloroform indicating that the product is not monomeric captopril or captopril lactone.

GPC analysis of the crude mixture in THF indicated a very high molecular weight component using the RALS detector. GPC of the purified solid in chloroform suggested the formation of a large amount of low molecular weight polymer with the average molecular weight correlating to approximately octameric captopril.

¹H NMR analysis of the purified solid in CDCl₃ again indicated preservation of the core captopril structure.

IR analysis revealed a spectrum similar to that of the monomeric lactone except for a small peak at 2539 cm⁻¹ assigned to SH and a weak peak at 1734 cm⁻¹ assigned to the CO═O of an acid, both consistent with the formation of polymer end groups.

DSC analysis gave a Tₘ of 96° C. and S_{olidify} of 9° C. (a shift from 107° C. and 32° C. respectively).

UV analysis showed a λₘₐₓ of 264 nm which is different to that of both captopril at 244 nm and captopril lactone at 249 nm. The polymer UV absorption peak was also much broader and similar to that obtained from other polymers.

Combined this data indicates a species chemically similar to the monomeric lactone but with a different molecular weight, solubility, melting point and λₘₐₓ. It is consistent with the formation of low molecular weight polycaptopril.

### Example 3

### Stability Studies

Samples of the purified solid from each of examples 1 and 2 were dissolved in DMSO and then separate aliquots were diluted in five different media (pH 7.4 buffer solution, pH 12.8 buffer solution, plasma, simulated intestinal fluid of pH 6.8 and simulated gastric fluid of pH 1.2). The ten resultant solutions were left for 16 hours and then analysed for the release of captopril monomer.

Within the limits of experimental error, no release of the captopril monomer was observed after 16 hours in any of the ten solutions. This demonstrates that polycaptopril has a good stability profile with hydrolysis occurring at a sufficiently slow rate to allow for the sustained controlled release of captopril in vivo.

1.-128. (canceled)

129. A polymer comprising a unit of formula I: 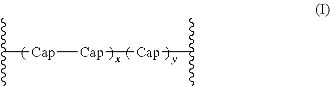
wherein x is an integer ≧1, y is 0 or 1, and -Cap- is a unit of formula II:

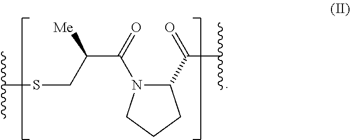

130. A polymer as claimed in claim 129, wherein the polymer is a homo-polymer.

131. A polymer as claimed in claim 130, wherein:
(i) the polymer has the formula Ia: 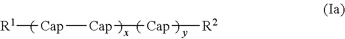
wherein R¹ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
R² is —R³, —OR³, —SR³ or —N(R³)₂, wherein each R³ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R³ groups together with the atom or atoms to which they are attached may form a cyclic hydrocarbyl group which may optionally be substituted and which may optionally include one or more heteroatoms N, O or S in its carbon skeleton; or
(ii) the polymer has the formula Ia: 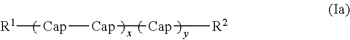
wherein R¹ is hydrogen and R² is —OH.

132. A polymer as claimed in claim 130, wherein:
(i) x is an integer from 1 to 100,000; or
(ii) x is an integer from 10 to 10,000; or
(iii) x is an integer from 100 to 1,000.

133. A polymer as claimed in claim 129, wherein the polymer is a copolymer formed with one or more additional polymeric substances.

134. A polymer as claimed in claim 133, wherein:
(i) the copolymer is a periodic copolymer, a random copolymer or a block copolymer; and/or
(ii) one or more of the additional polymeric substances is a biodegradable polymer, such as a polymer selected from the group consisting of poly-lactic acids, poly-lactides, poly(lactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof; and/or
(iii) one or more of the additional polymeric substances is a non-biodegradable polymer, such as a polymer selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof; and/or
(iv) x is an integer from 1 to 1,000, or from 2 to 100, or from 3 to 10.

135. A polymer comprising a unit of formula III within the polymer backbone: 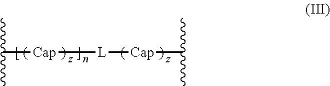
wherein n is an integer ≧1, each z is independently an integer ≧1, each L is independently any linking atom or group, and -Cap- is a unit of formula II: 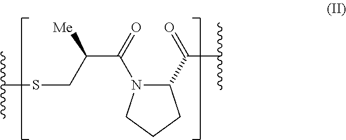
with the proviso that the unit of formula III does not comprise a -Cap-O— unit.

136. A polymer as claimed in claim 135, wherein:
(i) the polymer backbone does not comprise a -Cap-O— unit other than at a terminal position; and/or
(ii) the polymer backbone and/or the unit of formula III does not comprise a —COO— group within 2 bonds of a -Cap- unit other than at a terminal position; and/or
(iii) the polymer backbone and/or the unit of formula III does not comprise a —COO— group other than at a terminal position; and/or
(iv) each bond within the polymer backbone and/or the unit of formula III is more resistant to hydrolysis than the ester bond in H-Cap-OEt; and/or
(v) each -Cap- unit within the polymer backbone and/or the unit of formula III, other than a terminal -Cap- unit, is part of a -Cap-S— unit; and/or
(vi) at least one -Cap- unit is not at a terminal position; and/or
(vii) the polymer has the formula Ma: 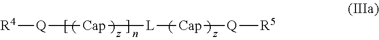
wherein each Q is independently L or a chemical bond;
R⁴ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
R⁵ is —R⁶, —OR⁶, —SR⁶ or —N(R⁶)₂, wherein each R⁶ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R⁶ groups together with the atom or atoms to which they are attached may form a cyclic hydrocarbyl group which may optionally be substituted and which may optionally include one or more heteroatoms N, O or S in its carbon skeleton; and/or
(viii) the polymer has the formula Ma: 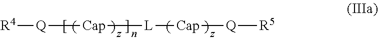
wherein each Q is independently L or a chemical bond, wherein R⁴ is hydrogen and R⁵ is —OH; and/or
(ix) n is an integer from 1 to 100,000, or from 10 to 10,000, or from 100 to 1,000; and/or
(x) each z is independently an integer from 1 to 100, or from Ito 10, or each z is 1; and/or
(xi) each L has a chain length of from 1 to 100 chemical bonds, or from 2 to 20 chemical bonds, or from 3 to 6 chemical bonds; and/or
(x) each L comprises one or more monomer units from a biodegradable polymer, such as a polymer selected from the group consisting of poly-lactic acids, poly-lactides, poly(lactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof; and/or
(xi) each L comprises one or more monomer units from a non-biodegradable polymer, such as a polymer selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

137. A polymer as claimed in claim 135, wherein each L has the formula Mb: 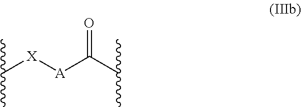
wherein X is —S— or —NR⁷—;
A is a chemical bond or an alkylene, alkenylene, alkynylene, arylene, arylalkylene, arylalkenylene, arylalkynylene, alkylarylene, alkenylarylene or alkynylarylene group, each of which may optionally be substituted, and each of which may optionally include one or more heteroatoms N, O or S in its carbon skeleton;
R⁷ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
wherein R⁷ and any substituent of A together with the atoms to which they are attached may form a cyclic hydrocarbyl group which may optionally be substituted and which may optionally include one or more additional hetero atoms N, O or S in its carbon skeleton.

138. A polymer comprising one or more base polymers and one or more cross-linking groups wherein at least one cross-linking group comprises one or more -Cap- units of formula II

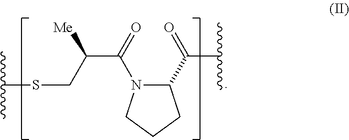

139. A polymer as claimed in claim 138, wherein:
(i) the cross-linking group comprises from 1 to 10-Cap- units, or comprises 1, 2 or 3-Cap- units; and/or
(ii) the cross-linking group contains only -Cap- units.

140. A polymer as claimed in claim 138, wherein the cross-linking group further comprises one or more linking groups L′, wherein each L′ is independently any atom or group.

141. A polymer as claimed in claim 140, wherein:
(i) the cross-linking group has the formula IV: 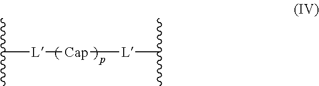
wherein p is an integer ≧1, or wherein p is an integer from 1 to 10, or wherein p is 1, 2 or 3; and/or
(ii) each L′ has a chain length of 1 to 100 chemical bonds, or a chain length of 2 to 20 chemical bonds, or a chain length of 3 to 6 chemical bonds; and/or
(iii) each L′ comprises one or more monomer units from a biodegradable polymer, such as a polymer selected from the group consisting of poly-lactic acids, poly-lactides, poly(lactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof; and/or
(iv) each L′ comprises one or more monomer units from a non-biodegradable polymer, such as a polymer selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof; and/or
(v) each L′ has the formula IVa: 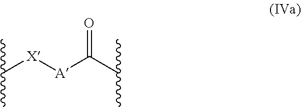
wherein X′ is —O—, —S— or —NR⁸—;
A′ is a chemical bond or an alkylene, alkenylene, alkynylene, arylene, arylalkylene, arylalkenylene, arylalkynylene, alkylarylene, alkenylarylene or alkynylarylene group, each of which may optionally be substituted, and each of which may optionally include one or more heteroatoms N, O or S in its carbon skeleton;
R⁸ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more hetero atoms N, O or S in its carbon skeleton; and
wherein R⁸ and any substituent of A′ together with the atoms to which they are attached may form a cyclic hydrocarbyl group which may optionally be substituted and which may optionally include one or more additional heteroatoms N, O or S in its carbon skeleton.

142. A polymer as claimed in claim 138, wherein:
(i) one or more of the base polymers are biodegradable polymers, such as polymers selected from the group consisting of poly-lactic acids, poly-lactides, poly(lactic acid-co-glycolic acids), poly(lactide-co-glycolides), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, cyclodextrins, polysaccharides, gelatin, collagen, albumin, fibrin, fibrinogen, polyketals, polyalkylene succinates, poly(malic acid), polypropylene oxides, and copolymers thereof; and/or
(ii) one or more of the base polymers are non-biodegradable polymers, such as polymers selected from the group consisting of ethyl celluloses, acrylates, methacrylates, pyrrolidones, polyoxyethylenes, polyoxyethylene-polypropylene copolymers, hydroxypropyl methyl celluloses, hydroxypropyl celluloses, methyl celluloses, polymethylmethacrylates, cellulose acetates and their derivatives, shellac, acrylic and methacrylic acid based polymers, and copolymers thereof.

143. A polymer as claimed in claim 129, wherein:
(i) said polymer is appended by or terminated with one or more groups selected from R⁹-Cap- and -Cap-R¹⁰,
wherein R⁹ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
R¹⁰ is —R¹¹, —OR¹¹, —SR¹¹ or —N(R¹¹)₂, wherein each R¹¹ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R¹¹ groups together with the atom or atoms to which they are attached may form a cyclic hydrocarbyl group which may optionally be substituted and which may optionally include one or more heteroatoms N, O or S in its carbon skeleton; and/or
(ii) said polymer is appended by or terminated with one or more groups selected from R⁹-Cap- and -Cap-R¹⁰, wherein R⁹ is hydrogen and R¹⁰ is —OH; and/or
(iii) said polymer further comprises one or more additional active pharmaceutical ingredients appended to, terminating or incorporated in the backbone of the polymer; and/or
(iv) said polymer is entirely biodegradable; and/or
(v) the decomposition products of said polymer are only active pharmaceutical ingredients and/or pharmaceutically acceptable decomposition products; and/or
(vi) said polymer has an average molecular weight of from 400 to 5,000,000, or from 1,000 to 1,000,000, or from 10,000 to 400,000.

144. A polymer as claimed in claim 135, wherein:
(i) said polymer is appended by or terminated with one or more groups selected from R⁹-Cap- and -Cap-R¹⁰,
wherein R⁹ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
R¹⁰ is R¹¹, —OR¹¹, —SR¹¹ or —N(R¹¹)₂, wherein each R¹¹ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R¹¹ groups together with the atom or atoms to which they are attached may form a cyclic hydrocarbyl group which may optionally be substituted and which may optionally include one or more heteroatoms N, O or S in its carbon skeleton; and/or
(ii) said polymer is appended by or terminated with one or more groups selected from R⁹-Cap- and -Cap-R¹⁰, wherein R⁹ is hydrogen and R¹⁰ is —OH; and/or
(iii) said polymer further comprises one or more additional active pharmaceutical ingredients appended to, terminating or incorporated in the backbone of the polymer; and/or
(iv) said polymer is entirely biodegradable; and/or
(v) the decomposition products of said polymer are only active pharmaceutical ingredients and/or pharmaceutically acceptable decomposition products; and/or
(vi) said polymer has an average molecular weight of from 400 to 5,000,000, or from 1,000 to 1,000,000, or from 10,000 to 400,000.

145. A polymer as claimed in claim 138, wherein:
(i) said polymer is appended by or terminated with one or more groups selected from R⁹-Cap- and -Cap-R¹⁰,
wherein R⁹ is hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
R¹⁰ is —R¹¹, —OR¹¹, —SR¹¹ or —N(R¹¹)₂, wherein each R¹¹ is independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two R¹¹ groups together with the atom or atoms to which they are attached may form a cyclic hydrocarbyl group which may optionally be substituted and which may optionally include one or more heteroatoms N, O or S in its carbon skeleton; and/or
(ii) said polymer is appended by or terminated with one or more groups selected from R⁹-Cap- and -Cap-R¹⁰, wherein R⁹ is hydrogen and R¹⁰ is —OH; and/or
(iii) said polymer further comprises one or more additional active pharmaceutical ingredients appended to, terminating or incorporated in the backbone of the polymer; and/or
(iv) said polymer is entirely biodegradable; and/or
(v) the decomposition products of said polymer are only active pharmaceutical ingredients and/or pharmaceutically acceptable decomposition products; and/or
(vi) said polymer has an average molecular weight of from 400 to 5,000,000, or from 1,000 to 1,000,000, or from 10,000 to 400,000.

146. A process for synthesising a polymer as claimed in claim 129, said process comprising the use of H-Cap-OH and/or 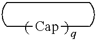
wherein q is an integer from 1 to 10, and/or protected derivatives thereof.

147. A process for synthesising a polymer as claimed in claim 135, said process comprising the use of H-Cap-OH and/or 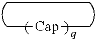
wherein q is an integer from 1 to 10, and/or protected derivatives thereof.

148. A process for synthesising a polymer as claimed in claim 138, said process comprising the use of H-Cap-OH and/or 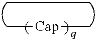
wherein q is an integer from 1 to 10, and/or protected derivatives thereof.

149. A process for synthesising a polymer, said process comprising:
(a) the condensation of a —COOH group of either H-Cap-OH or a protected derivative thereof with a —SH group of another molecule; and/or
(b) the condensation of a —C(O)S— group of either 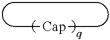
or a protected derivative thereof, wherein q is an integer from 1 to 10, with a —SH group of another molecule; and/or
(c) the condensation of a —SH group of either H-Cap-OH or a protected derivative thereof with a —COOH group of another molecule;
wherein -Cap- is a unit of formula II:

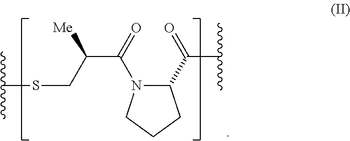

150. A process as claimed in claim 149, wherein:
(i) the condensation is between two molecules of H-Cap-OH, or protected and/or polymerised derivatives thereof; or
(ii) the condensation is between one molecule of H-Cap-OH, or a protected and/or polymerised derivative thereof, and one molecule of 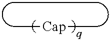
or a protected derivative thereof; or
(iii) the condensation is between two molecules of 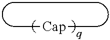
or protected derivatives thereof.

151. A process as claimed in claim 149, wherein the synthesis is achieved via:
(i) chemical activation of a —COOH group; and/or
(ii) enzymatic catalysis, for example wherein a lipase enzyme is used.

152. A polymer produced by a process according to claim 149.

153. A pharmaceutical composition or a medical product comprising a polymer as claimed in claim 129.

154. A pharmaceutical composition or a medical product comprising a polymer as claimed in claim 135.

155. A pharmaceutical composition or a medical product comprising a polymer as claimed in claim 138.

156. A pharmaceutical composition or a medical product comprising a polymer as claimed in claim 152.

157. A method for the treatment or prevention of collagen deposition, fibrosis, scars, burns, or unwanted tissue formation, comprising administering to a subject in need thereof a therapeutically or prophylactically effective amount of a polymer as claimed in claim 129.

158. A method for the treatment or prevention of collagen deposition, fibrosis, scars, burns, or unwanted tissue formation, comprising administering to a subject in need thereof a therapeutically or prophylactically effective amount of a polymer as claimed in claim 135.

159. A method for the treatment or prevention of collagen deposition, fibrosis, scars, burns, or unwanted tissue formation, comprising administering to a subject in need thereof a therapeutically or prophylactically effective amount of a polymer as claimed in claim 138.

160. A method for the treatment or prevention of collagen deposition, fibrosis, scars, burns, or unwanted tissue formation, comprising administering to a subject in need thereof a therapeutically or prophylactically effective amount of a polymer as claimed in claim 152.

161. A polymer comprising one or more -Cap- units of formula II: 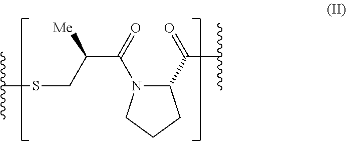
for use in the treatment or prevention of collagen deposition, fibrosis, scars, burns, or unwanted tissue formation.

162. A method for the treatment or prevention of collagen deposition, fibrosis, scars, burns, or unwanted tissue formation, comprising administering to a subject in need thereof a therapeutically or prophylactically effective amount of a polymer comprising one or more -Cap- units of formula II:

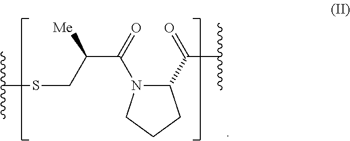

163. A polymer as claimed in claim 129, wherein each -Cap- unit is instead independently defined as any unit such that H-Cap-OH or a pharmaceutically acceptable salt or derivative thereof is capable of inhibiting an angiotensin converting enzyme.

164. A polymer as claimed in claim 163, wherein:
(i) each -Cap- unit is the same, or the polymer contains two or more structurally different types of -Cap- unit; and/or
(ii) at least one type of -Cap- unit is defined such that H-Cap-OH or a pharmaceutically acceptable salt or derivative thereof is capable of inhibiting an angiotensin-I converting enzyme; and/or
(iii) at least one type of -Cap- unit is defined such that H-Cap-OH or a pharmaceutically acceptable salt or derivative thereof is capable of inhibiting vasopeptidase; and/or
(iv) at least one type of -Cap- unit is defined such that H-Cap-OH comprises a thiol group and a carboxylic acid group; and/or
(v) at least one type of -Cap- unit is defined as a unit of formula IIa: 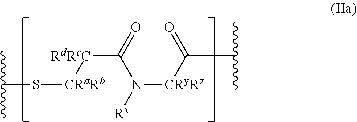
wherein R^{a}, R^{b}, R^{c}, R^{d}, R^{x}, R^{y} and R^{z} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y} and R^{z} together with the atom or atoms to which they are attached may form a cyclic hydrocarbyl group which may optionally be substituted and which may optionally include one or more heteroatoms N, O or S in its carbon skeleton; and/or
(vi) at least one type of -Cap- unit is defined as a unit of formula IIc: 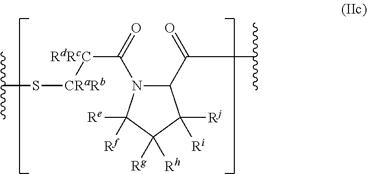
wherein R^{a}, R^{b}, R^{c}, R^{d}, R^{e}, R^{f}, R^{g}, R^{h}, Rⁱ and R^{j} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{h}, R^{h}, Rⁱ and R^{j} together with the atom or atoms to which they are attached may form a cyclic hydrocarbyl group which may optionally be substituted and which may optionally include one or more heteroatoms N, O or S in its carbon skeleton; and/or
(vii) at least one type of -Cap- unit is defined as a unit of formula 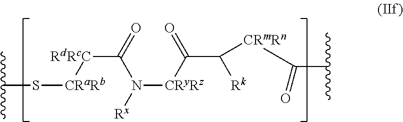
wherein R^{a}, R^{b}, R^{c}, R^{d}, R^{x}, R^{y}, R^{z}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y}, R^{z}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(viii) at least one type of -Cap- unit is defined as a unit of formula IIg: 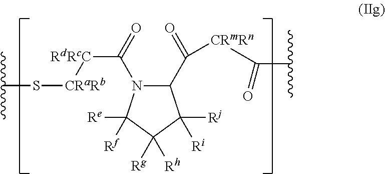
wherein R^{a}, R^{b}, R^{c}, R^{d}, R^{e}, R^{f}, R^{g}, R^{h}, Rⁱ, R^{j}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, Rⁱ, R^{j}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(ix) at least one type of -Cap- unit is defined as a unit of formula IIj: 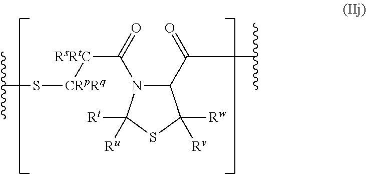
wherein R^{P}, R^{q}, R^{r}, R^{s}, R^{t}, R^{u}, R^{v} and R^{w} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{P}, R^{q}, R^{r}, R^{s}, R^{t}, R^{u}, R^{v} and R^{w} together with the atom or atoms to which they are attached may form a cyclic hydrocarbyl group which may optionally be substituted and which may optionally include one or more heteroatoms N, O or S in its carbon skeleton; and/or
(x) at least one type of -Cap- unit is defined as a unit of formula IIe, IIi, IIm or IIn:

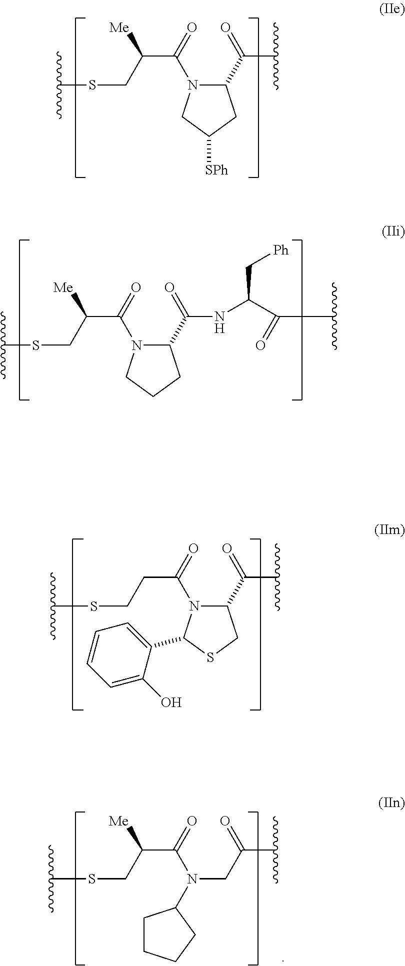

165. A polymer as claimed in claim 135, wherein each -Cap- unit is instead independently defined as any unit such that H-Cap-OH or a pharmaceutically acceptable salt or derivative thereof is capable of inhibiting an angiotensin converting enzyme.

166. A polymer as claimed in claim 165, wherein:
(i) each -Cap- unit is the same, or the polymer contains two or more structurally different types of -Cap- unit; and/or
(ii) at least one type of -Cap- unit is defined such that H-Cap-OH or a pharmaceutically acceptable salt or derivative thereof is capable of inhibiting an angiotensin-I converting enzyme; and/or
(iii) at least one type of -Cap- unit is defined such that H-Cap-OH or a pharmaceutically acceptable salt or derivative thereof is capable of inhibiting vasopeptidase; and/or
(iv) at least one type of -Cap- unit is defined such that H-Cap-OH comprises a thiol group and a carboxylic acid group; and/or
(v) at least one type of -Cap- unit is defined as a unit of formula IIa: 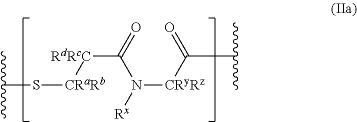
wherein R^{a}, R^{b}, R^{c}, R^{d}, R^{x}, R^{y} and R^{z} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y} and R^{z} together with the atom or atoms to which they are attached may form a cyclic hydrocarbyl group which may optionally be substituted and which may optionally include one or more heteroatoms N, O or S in its carbon skeleton; and/or
(vi) at least one type of -Cap- unit is defined as a unit of formula IIc: 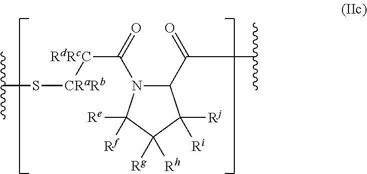
wherein R^{a}, R^{b}, R^{c}, R^{d}, R^{e}, R^{f}, R^{g}, R^{h}, Rⁱ and R^{j} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, Rⁱ and R^{j} together with the atom or atoms to which they are attached may form a cyclic hydrocarbyl group which may optionally be substituted and which may optionally include one or more hetero atoms N, O or S in its carbon skeleton; and/or
(vii) at least one type of -Cap- unit is defined as a unit of formula IIf: 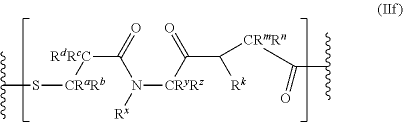
wherein R^{a}, R^{b}, R^{c}, R^{d}, R^{x}, R^{y}, R^{z}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y}, R^{z}, R^{k}, R^{m} and Rⁿ together with the atom or atoms to which they are attached may form a cyclic hydrocarbyl group which may optionally be substituted and which may optionally include one or more hetero atoms N, O or S in its carbon skeleton; and/or
(viii) at least one type of -Cap- unit is defined as a unit of formula IIg: 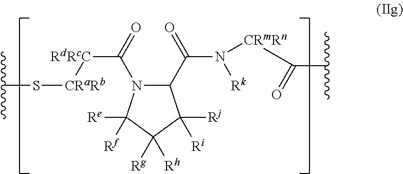
wherein R^{a}, R^{b}, R^{c}, R^{d}, R^{e}, R^{f}, R^{g}, R^{h}, Rⁱ, R^{j}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, Rⁱ, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(ix) at least one type of -Cap- unit is defined as a unit of formula IIj: 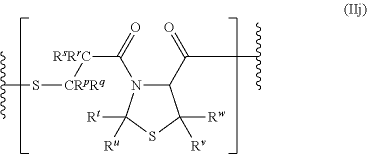
wherein R^{P}, R^{q}, R^{r}, R^{s}, R^{t}, R^{u}, R^{v} and R^{w} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{P}, R^{q}, R^{r}, R^{s}, R^{t}, R^{u}, R^{v} and R^{w} together with the atom or atoms to which they are attached may form a cyclic hydrocarbyl group which may optionally be substituted and which may optionally include one or more heteroatoms N, O or S in its carbon skeleton; and/or
(x) at least one type of -Cap- unit is defined as a unit of formula IIe, IIi, IIm or IIn:

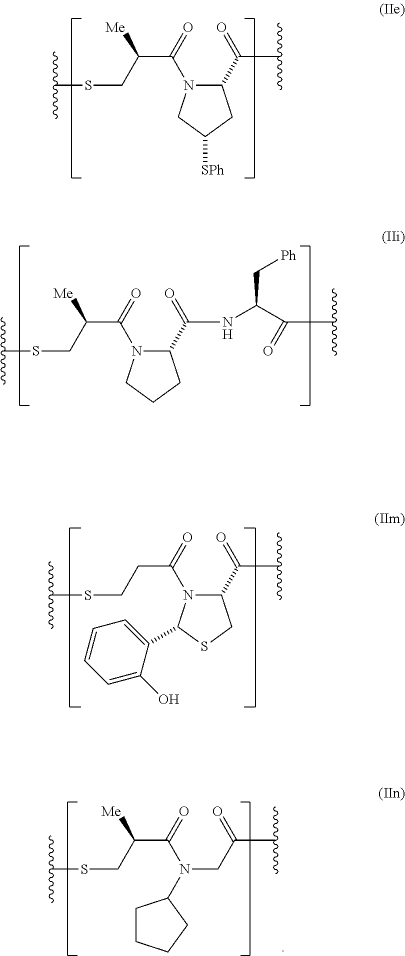

167. A polymer as claimed in claim 138, wherein each -Cap- unit is instead independently defined as any unit such that H-Cap-OH or a pharmaceutically acceptable salt or derivative thereof is capable of inhibiting an angiotensin converting enzyme.

168. A polymer as claimed in claim 167, wherein:
(i) each -Cap- unit is the same, or the polymer contains two or more structurally different types of -Cap- unit; and/or
(ii) at least one type of -Cap- unit is defined such that H-Cap-OH or a pharmaceutically acceptable salt or derivative thereof is capable of inhibiting an angiotensin-I converting enzyme; and/or
(iii) at least one type of -Cap- unit is defined such that H-Cap-OH or a pharmaceutically acceptable salt or derivative thereof is capable of inhibiting vasopeptidase; and/or
(iv) at least one type of -Cap- unit is defined such that H-Cap-OH comprises a thiol group and a carboxylic acid group; and/or
(v) at least one type of -Cap- unit is defined as a unit of formula IIa: 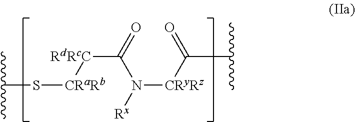
wherein R^{a}, R^{b}, R^{c}, R^{d}, R^{x}, R^{y} and R^{z} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y} and R^{z} together with the atom or atoms to which they are attached may form a cyclic hydrocarbyl group which may optionally be substituted and which may optionally include one or more heteroatoms N, O or S in its carbon skeleton; and/or
(vi) at least one type of -Cap- unit is defined as a unit of formula IIc: 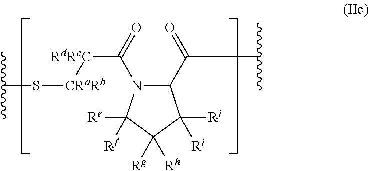
wherein R^{a}, R^{b}, R^{c}, R^{d}, R^{e}, R^{f}, R^{g}, R^{h}, RRⁱ and R^{j} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, RRⁱ and R^{j} together with the atom or atoms to which they are attached may form a cyclic hydrocarbyl group which may optionally be substituted and which may optionally include one or more heteroatoms N, O or S in its carbon skeleton; and/or
(vii) at least one type of -Cap- unit is defined as a unit of formula 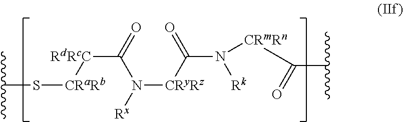
wherein R^{a}, R^{b}, R^{c}, R^{d}, R^{x}, R^{y}, R^{z}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y}, R^{z}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(viii) at least one type of -Cap- unit is defined as a unit of formula IIg: 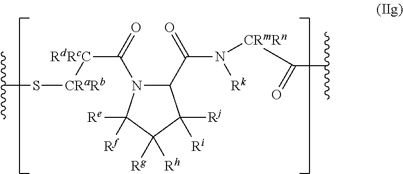
wherein R^{a}, R^{b}, R^{c}, R^{d}, R^{e}, R^{f}, R^{g}, R^{h}, Rⁱ, R^{j}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, Rⁱ, R^{j}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(ix) at least one type of -Cap- unit is defined as a unit of formula IIi: 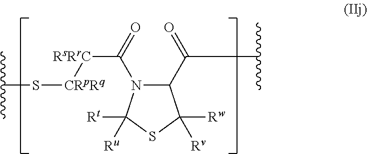
wherein R^{p}, R^{q}, R^{r}, R^{s}, R^{t}, R^{u}, R^{v} and R^{w} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{P}, R^{q}, R^{r}, R^{s}, R^{t}, R^{u}, R^{v} and R^{w} together with the atom or atoms to which they are attached may form a cyclic hydrocarbyl group which may optionally be substituted and which may optionally include one or more heteroatoms N, O or S in its carbon skeleton; and/or
(x) at least one type of -Cap- unit is defined as a unit of formula IIe, IIi, IIm or IIn:

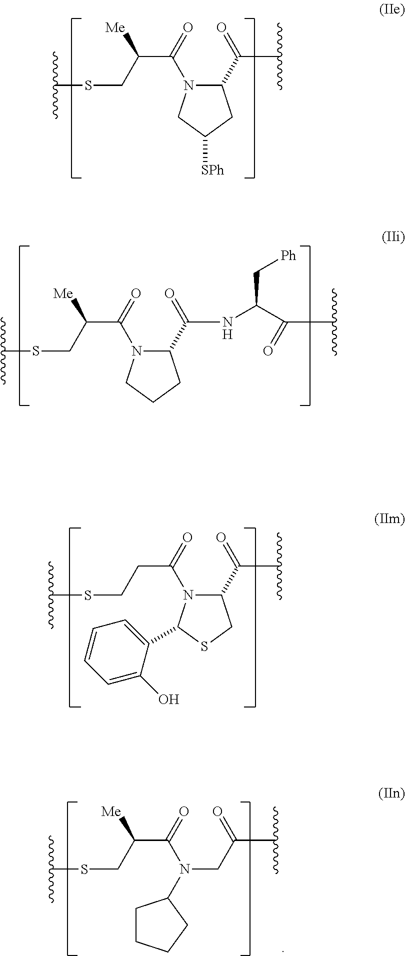

169. A process as claimed in claim 149, wherein each -Cap- unit is instead independently defined as any unit such that H-Cap-OH or a pharmaceutically acceptable salt or derivative thereof is capable of inhibiting an angiotensin converting enzyme.

170. A process as claimed in claim 169, wherein:
(i) each -Cap- unit is the same, or the polymer contains two or more structurally different types of -Cap- unit; and/or
(ii) at least one type of -Cap- unit is defined such that H-Cap-OH or a pharmaceutically acceptable salt or derivative thereof is capable of inhibiting an angiotensin-I converting enzyme; and/or
(iii) at least one type of -Cap- unit is defined such that H-Cap-OH or a pharmaceutically acceptable salt or derivative thereof is capable of inhibiting vasopeptidase; and/or
(iv) at least one type of -Cap- unit is defined such that H-Cap-OH comprises a thiol group and a carboxylic acid group; and/or
(v) at least one type of -Cap- unit is defined as a unit of formula IIa: 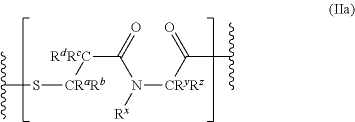
wherein R^{a}, R^{b}, R^{c}, R^{d}, R^{x}, R^{y} and R^{z} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{x}, R^{y} and R^{z} together with the atom or atoms to which they are attached may form a cyclic hydrocarbyl group which may optionally be substituted and which may optionally include one or more heteroatoms N, O or S in its carbon skeleton; and/or
(vi) at least one type of -Cap- unit is defined as a unit of formula IIc: 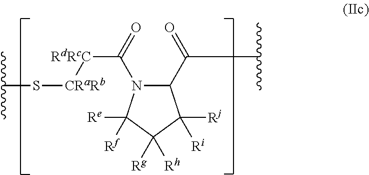
wherein R^{a}, R^{b}, R^{c}, R^{d}, R^{e}, R^{f}, R^{g}, R^{h}, Rⁱ and R^{j} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, Rⁱ and R^{j} together with the atom or atoms to which they are attached may form a cyclic hydrocarbyl group which may optionally be substituted and which may optionally include one or more heteroatoms N, O or S in its carbon skeleton; and/or
(vii) at least one type of -Cap- unit is defined as a unit of formula IIf: 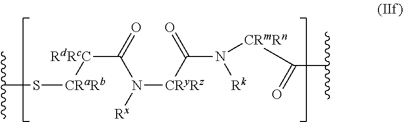
wherein R^{a}, R^{b}, R^{c}, R^{d}, R^{x}, R^{y}, R^{z}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more hetero atoms N, O or S in its carbon skeleton, and wherein any two or more of R^{a}, R^{b}, R^{c}, R^{d}, R^{x}, R^{y}, R^{z}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(viii) at least one type of -Cap- unit is defined as a unit of formula IIg: 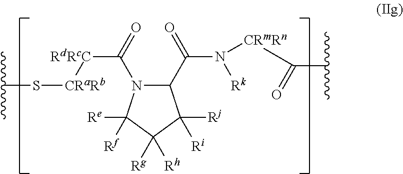
wherein R^{a}, R^{b}, R^{c}, R^{d}, R^{e}, R^{f}, R^{g}, R^{h}, Rⁱ, R^{j}, R^{k}, R^{m} and Rⁿ are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{a}, R^{b}, R^{c}, R^{d}, R^{e}, R^{f}, R^{g}, R^{h}, Rⁱ, R^{j}, R^{k}, R^{m} and Rⁿ together with the atom or atoms to which they are attached may form a cyclic hydrocarbyl group which may optionally be substituted and which may optionally include one or more heteroatoms N, O or S in its carbon skeleton; and/or
(ix) at least one type of -Cap- unit is defined as a unit of formula IIj: 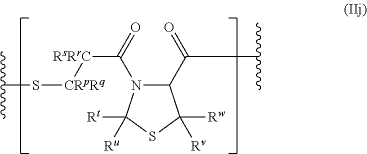
wherein R^{P}, R^{q}, R^{r}, R^{s}, R^{t}, R^{u}, R^{v} and R^{w} are each independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, and wherein any two or more of R^{P}, R^{q}, R^{r}, R^{s}, R^{t}, R^{u}, R^{v} and R^{w} together with the atom or atoms to which they are attached may form a cyclic hydrocarbyl group which may optionally be substituted and which may optionally include one or more heteroatoms N, O or S in its carbon skeleton; and/or
(x) at least one type of -Cap- unit is defined as a unit of formula IIe, IIi, IIm or IIn: [chemical structure]
